(19) United States
(12) Patent Application Publication
(10) Pub. No.: US 2014/0199286 A1
(43) Pub. Date: Jul. 17, 2014

(54) LYOPHILIZATION PROCESS

(71) Applicants: Qinghai Zhao, North Potomac, MD (US); Xia Luo, Rockville, MD (US); Jason Bock, North Potomac, MD (US)

(72) Inventors: Qinghai Zhao, North Potomac, MD (US); Xia Luo, Rockville, MD (US); Jason Bock, North Potomac, MD (US)

(73) Assignee: TEVA PHARMACEUTICAL INDUSTRIES, LTD., Petach-Tikva (IL)

(21) Appl. No.: 14/155,056

(22) Filed: Jan. 14, 2014

Related U.S. Application Data
(60) Provisional application No. 61/752,797, filed on Jan. 15, 2013.
(60) Provisional application No. 61/784,538, filed on Mar. 14, 2013.

(51) Int. Cl. A61J 3/00; A61K 38/38; A61K 38/46

(52) U.S. Cl. CPC A61J 3/00; A61K 38/465; A61K 38/385
USPC 424/94.6; 435/197; 53/467

(57) ABSTRACT

The present invention provides a process for producing a lyophilized pharmaceutical composition containing a protein. The present invention further provides a product produced by the process. The present invention further provides a process for producing an injectable pharmaceutical composition. The present invention further provides a method of treating a patient with a therapeutic protein composition.

This application claims the benefit of U.S. Provisional Application Nos. 61/752,797, filed Jan. 15, 2013, and 61/784,538, filed Mar. 14, 2013, the contents of which are hereby incorporated by reference in their entirety.

Throughout this application, various publications are referenced by author and publication date. Full citations for these publications may be found at the end of the specification immediately preceding the claims. The disclosures of these publications are hereby incorporated by reference into this application to describe more fully the art to which this invention pertains.

### BACKGROUND OF THE INVENTION

Lyophilization is widely used to produce and distribute pharmaceutical products, including proteins. However, as the concentration of protein in a lyophilate increases, the time required to reconstitute it increases as well.

### SUMMARY OF THE INVENTION

The present invention provides a process for producing a lyophilized pharmaceutical composition containing a protein, comprising the steps of:
(i) obtaining a solution comprising the protein in one or more containers;
(ii) placing the one or more containers within a chamber of a lyophilizing unit;
(iii) reducing the temperature to an initial freezing temperature of −60° C. to −25° C. at a rate of 0.2° C. to 2.0° C. per minute, and holding the temperature at the initial freezing temperature for 1 to 6 hours to form a frozen solution;
(iv) increasing the temperature to an annealing temperature of the frozen solution of −30° C. to −10° C. at a rate of 0.2 to 2.0° C. per minute, and holding the temperature at the annealing temperature for 1 to 10 hours;
(v) reducing the temperature to a refreezing temperature of −60 to −25 at a rate of 0.2 to 2.0° C. per minute, and holding the temperature at the refreezing temperature for 1 to 6 hours;
(vi) reducing the pressure of the chamber to 50 to 500 mT, and continuing to hold the temperature at the refreezing temperature for an additional 0 to 4 hours;
(vii) increasing the temperature to a primary drying temperature of −30 to −10° C. at a rate of 0.2 to 2.0° C. per minute, and holding the temperature at the primary drying temperature for 10 to 72 hours;
(viii) increasing the temperature to a secondary drying temperature of 5 to 30° C. at a rate of 0.2 to 2.0° C. per minute, and holding the temperature at the secondary drying temperature for 2 to 25 hours; and
(ix) increasing the pressure of the chamber to partial atmospheric pressure.

The present invention further provides a product produced by the process.

The present invention further provides a process for producing an injectable pharmaceutical composition, comprising obtaining an amount of the lyophilized pharmaceutical composition comprising a protein produced by the process, and reconstituting the lyophilized pharmaceutical composition with water for injection within 15 minutes, thereby producing an injectable pharmaceutical composition.

The present invention further provides a method of treating a patient with a therapeutic protein composition, comprising obtaining an amount of the lyophilized pharmaceutical composition comprising a protein produced, reconstituting the lyophilized pharmaceutical composition with water for injection within 15 minutes to form a reconstituted solution, and administering the reconstituted solution to the patient, thereby treating the patient.

### DETAILED DESCRIPTION OF THE INVENTION

As used herein, and unless stated otherwise, each of the following terms shall have the definition set forth below.

As used herein, “reconstituted solution” means a solution produced by dissolving a lyophilized substance in an amount of solvent. In an embodiment, the solvent is water for injection (WFI). In an embodiment, the volume of solvent used is the volume of pre-lyophilization solution used to make the lyophilized substance. In an embodiment, the volume of solvent used is more than the volume of pre-lyophilization solution used to make the lyophilized substance. In an embodiment, the volume of solvent used is 90 percent of than the volume of pre-lyophilization solution used to make the lyophilized substance. In an embodiment, the volume of solvent used is less than the volume of pre-lyophilization solution used to make the lyophilized substance.

As used herein, “purity,” as in purity of a pharmaceutical composition, refers to the relative amount of a protein that is not disintegrated, monomeric, and in its native conformation. Purity may be measured by size exclusion high performance liquid chromatography (SE-HPLC), hydrophobic interaction high performance liquid chromatography (HI-HPLC), sodium dodecylsylfate polyacramide gel electrophoresis (SDS-PAGE), or any other method known in the art, and may be expressed as a percentage. As used herein, “recommended conditions,” or “recommended storage conditions” as in a sample stored at the recommended conditions, means the storage conditions determined to keep the characteristics of the composition within acceptable parameters for the duration of storage. In a specific embodiment, the recommended storage conditions are a temperature of 2-8° C., in an upright position, and/or with limited exposure to light.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49 mg unit amounts are included as embodiments of this invention.

The specific embodiments and examples described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

The present invention provides a process for producing a lyophilized pharmaceutical composition containing a protein, comprising the steps of:
(i) obtaining a solution comprising the protein in one or more containers;
(ii) placing the one or more containers within a chamber of a lyophilizing unit;
(iii) reducing the temperature to an initial freezing temperature of −60° C. to −25° C. at a rate of 0.2° C. to 2.0° C. per minute, and holding the temperature at the initial freezing temperature for 1 to 6 hours to form a frozen solution;
(iv) increasing the temperature to an annealing temperature of the frozen solution of −30° C. to −10° C. at a rate of 0.2 to 2.0° C. per minute, and holding the temperature at the annealing temperature for 1 to 10 hours;
(v) reducing the temperature to a refreezing temperature of −60 to −25 at a rate of 0.2 to 2.0° C. per minute, and holding the temperature at the refreezing temperature for 1 to 6 hours;
(vi) reducing the pressure of the chamber to 50 to 500 mT, and continuing to hold the temperature at the refreezing temperature for an additional 0 to 4 hours;
(vii) increasing the temperature to a primary drying temperature of −30 to −10° C. at a rate of 0.2 to 2.0° C. per minute, and holding the temperature at the primary drying temperature for 10 to 72 hours;
(viii) increasing the temperature to a secondary drying temperature of 5 to 30° C. at a rate of 0.2 to 2.0° C. per minute, and holding the temperature at the secondary drying temperature for 2 to 25 hours; and
(ix) increasing the pressure of the chamber to partial atmospheric pressure.

In an embodiment, step (ii) placing the containers within the chamber of the lyophilizing unit comprises placing the containers on a shelf which is at an initial shelf temperature of from −40 to 10° C. within the chamber and holding the temperature of the shelf at the initial shelf temperature for 0 to 5 hours before initiating step (iii).

In an embodiment, step (ii) placing the containers within the chamber of the lyophilizing unit comprises placing the containers on a shelf which is at an initial shelf temperature of from −40 to 5° C. within the chamber and holding the temperature of the shelf at the initial shelf temperature for 0 to 5 hours before initiating step (iii).

In an embodiment, the initial shelf temperature is from −5 to 10° C. In an embodiment, the initial shelf temperature is −5° C., −4° C., −3° C., −2° C., −1° C., 0° C., 1° C., 2° C., 3° C., 4° C., 5° C., 6° C., 7° C., 8° C., 9° C. or 10° C.

In an embodiment, the initial shelf temperature is from −5 to 5° C. In an embodiment, the initial shelf temperature is −5° C., −4° C., −3° C., −2° C., −1° C., 0° C., 1° C., 2° C., 3° C., 4° C. or 5° C.

In an embodiment, the shelf is held at the initial shelf temperature for 1.1 to 5 hours. In an embodiment, the shelf is held at the initial shelf temperature for 2 to 5 hours. In an embodiment, the shelf is held at the initial shelf temperature for 2, 3, 4 or 5 hours.

In an embodiment, the shelf is held at the initial shelf temperature for 2 hours or more. In an embodiment, the shelf is held at the initial shelf temperature for 3 to 5 hours.

In an embodiment, the temperature in steps (iii) to (viii) is the shelf temperature. In an embodiment, the temperature in steps (iii) to (viii) is the chamber temperature.

In an embodiment, step (ii) further comprises pre-cooling the one or more containers. In an embodiment, the pre-cooling is by liquid nitrogen.

In an embodiment, the containers are pre-cooled to a temperature from −5 to 5° C. In an embodiment, the containers are pre-cooled to −5° C., −4° C., −3° C., −2° C., −1° C., 0° C., 1° C., 2° C., 3° C., 4° C., 5° C., 6° C., 7° C., 8° C., 9° C. or 10° C.

In an embodiment, in step (iii) the temperature is reduced at a rate of 0.3° C. per minute. In an embodiment, in step (iii) the temperature is reduced at a rate of 0.2° C. per minute. In an embodiment, in step (iii) the temperature is reduced at a rate of 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C. or 2.0° C. per minute.

In an embodiment, in step (iii) the temperature is held at the initial freezing temperature for 2.1 to 6 hours. In an embodiment, in step (iii) the temperature is held at the initial freezing temperature for 2.5 to 6 hours. In an embodiment, in step (iii) the temperature is held at the initial freezing temperature for 2, 2.1, 2.5, 3, 3.5, 4, 4.5, 5, 5.5 or 6 hours. In an embodiment, in step (iii) the temperature is held at the initial freezing temperature for more than 2, 2.1, 2.5, 3, 3.5, 4, 4.5, 5 or 5.5 hours.

In an embodiment, in step (iv) the temperature is increased at a rate of 0.8° C. per minute. In an embodiment, in step (iv) the temperature is increased at a rate of 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C. or 2.0° C. per minute.

In an embodiment, in step (iv) the temperature is held at the annealing temperature for 2.1 to 10 hours. In an embodiment, in step (iv) the temperature is held at the annealing temperature for 3 to 10 hours. In an embodiment, in step (iv) the temperature is held at the annealing temperature for 5 to 10 hours. In an embodiment, in step (iv) the temperature is held at the annealing temperature for 1, 1.5, 2, 2.1, 2.5, 3, 3.5, 4, 4.5, 5, 5.5, 6, 6.5, 7, 7.5, 8, 8.5, 9, 9.5 or 10 hours. In an embodiment, in step (iv) the temperature is held at the annealing temperature for more than 1, 1.5, 2, 2.1, 2.5, 3, 3.5, 4, 4.5, 5, 5.5, 6, 6.5, 7, 7.5, 8, 8.5, 9 or 9.5 hours.

In an embodiment, in step (iv) the temperature is held at the annealing temperature for 5 hours.

In an embodiment, in step (v) the temperature is reduced at a rate of 0.3° C. per minute.

In an embodiment, in step (v) the temperature is held at the refreezing temperature for 1.1 to 6 hours. In an embodiment, in step (v) the temperature is held at the refreezing temperature for 2 to 6 hours. In an embodiment, in step (v) the temperature is held at the refreezing temperature for 2, 2.1, 2.5, 3, 3.5, 4, 4.5, 5, 5.5 or 6 hours. In an embodiment, in step (v) the temperature is held at the refreezing temperature for more than 2, 2.1, 2.5, 3, 3.5, 4, 4.5, 5 or 5.5 hours.

In an embodiment, in step (vi) the temperature is held at the refreezing temperature for 1 hour.

In an embodiment, in step (vii) the temperature is increased at a rate of 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C. or 2.0° C. per minute.

In an embodiment, in step (vii) the temperature is held at the primary drying temperature for 36 hours or more.

In an embodiment, in step (vii) the temperature is held at the primary drying temperature for 36 hours.

In an embodiment, in step (vii) the temperature is held at the primary drying temperature for 10 to 29 hours.

In an embodiment, in step (vii) the temperature is held at the primary drying temperature for 29 to 42 hours.

In an embodiment, in step (vii) the primary drying temperature is −30° C. to −5° C.

In an embodiment, the process further comprises measuring the temperature of the frozen solution within one or more of the containers during step (vii), wherein in step (vii) the temperature is held at the primary drying temperature for three hours beyond the time at which the temperature of each measured container is equal to or greater than the primary drying temperature.

In an embodiment, in step (viii) the temperature is increased at a rate of 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C. or 2.0° C. per minute.

In an embodiment, in step (viii) the temperature is held at the secondary drying temperature for 4.1, 5, 6, 7, 8, 9, 10, 11, 12, 13, 14 or 15 or more hours.

In an embodiment, in step (viii) the temperature is held at the secondary drying temperature for 15 hours.

In an embodiment, in step (ix) the partial atmospheric pressure is 810 mBar.

In an embodiment, in step (ix) the partial atmospheric pressure is 600 T.

In an embodiment, in step (ix) the restoring to partial atmospheric pressure is adding sterile filtered nitrogen to the chamber.

In an embodiment, the process further comprises the step:
(x) sealing the containers.

In an embodiment, the sealing comprises inserting a stopper.

In an embodiment, the initial freezing temperature is −49° C. to −25° C. In an embodiment, the initial freezing temperature is −47° C. to −40° C. In an embodiment, the initial freezing temperature is −45° C. to −35° C.

In an embodiment, the initial freezing temperature is −45° C.

In an embodiment, the annealing temperature is −19 to −10° C.

In an embodiment, the annealing temperature is −30 to −20° C.

In an embodiment, the annealing temperature is −25 to −15° C.

In an embodiment, the annealing temperature is −19 to −15° C.

In an embodiment, the annealing temperature is −15 to −10° C.

In an embodiment, the annealing temperature is −19° C., −18° C., −17° C., −16° C., −15° C., −14° C., −13° C., −12° C., −11° C. or −10° C.

In an embodiment, the refreezing temperature is −49 to −25° C.

In an embodiment, the refreezing temperature is −45° C. In an embodiment, the refreezing temperature is the same as the initial freezing temperature.

In an embodiment, the primary drying temperature is −19° C. to 0° C. In an embodiment, the primary drying temperature is −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C. or 0° C.

In an embodiment, the primary drying temperature is −10° C.

In an embodiment, the secondary drying temperature is 5 to 30° C. In an embodiment, the secondary drying temperature is 20° C. to 30° C.

In an embodiment, the secondary drying temperature is 25° C.

In an embodiment, in step (vi) the pressure is reduced to 100 mT.

In an embodiment, the solution comprising a protein has a protein concentration from 2 to 250 mg/ml.

In an embodiment, the solution comprising a protein has a protein concentration greater than 65 mg/ml.

In an embodiment, the solution comprising a protein has a protein concentration from 65 to 250 mg/ml. In an embodiment, the solution comprising a protein has a protein concentration from 80 to 120 mg/ml. In an embodiment, the solution comprising a protein has a protein concentration of 100, 150, 200, or 250 mg/ml.

In an embodiment, the solution comprising a protein has a protein concentration from 100 to 110 mg/ml.

In an embodiment, in step (i) each of the one or more containers contains from 0.5 to 2.0 ml of the solution.

In an embodiment, each of the one or more containers contains 1.0 to 1.2 ml of the solution.

In an embodiment, the process produces a lyophilized pharmaceutical composition containing a protein, which reconstitutes in water for injection in 15 minutes or less.

In an embodiment, the process produces a lyophilized pharmaceutical composition containing a protein, which reconstitutes in water for injection in 6 minutes or less.

In an embodiment, the process produces a lyophilized pharmaceutical composition containing a protein, which reconstitutes in water for injection after one month of storage at recommended conditions in 15 minutes or less.

In an embodiment, the process produces a lyophilized pharmaceutical composition containing a protein, which reconstitutes in water for injection after one month of storage at recommended conditions in 6 minutes or less.

In an embodiment, the solution comprising the protein further comprises 40 to 60 mM phosphate.

In an embodiment, the solution comprising the protein further comprises 100 to 150 mM mannitol, 20 to 40 mM trehalose, or 0.02 to 0.05 percent polysorbate 80.

In an embodiment, the solution comprising the protein further comprises one or more of 100 to 150 mM mannitol, 20 to 40 mM trehalose, or 0.02 to 0.05 percent polysorbate 80. In an embodiment, the solution comprising the protein comprises 50 mM sodium phosphate, 115 mM mannitol, 35 mM trehalose, and 0.03 percent polysorbate 80. In an embodiment, the solution comprising the protein comprises 60 mM sodium phosphate, 100 mM mannitol, 30 mM trehalose, and 0.03 percent polysorbate 80. In an embodiment, the sodium phosphate comprises 16 mM sodium phosphate monobasic and 34 mM sodium phosphate dibasic.

In an embodiment, the solution comprising the protein further comprises mannitol and trehalose in a molar ratio of about 3.3 to 1.

In an embodiment, the process produces a lyophilized pharmaceutical composition containing a protein, which has a residual moisture of 3.0 weight percent or less.

In an embodiment, the process produces a lyophilized pharmaceutical composition containing a protein, which has a residual moisture of 0.3 weight percent or less.

In an embodiment, the residual moisture is 3 percent or less.

In an embodiment, the residual moisture is 0.1, 0.3, 0.4, 0.5, 1 or 2 percent or less.

In an embodiment, the process produces a lyophilized pharmaceutical composition containing a protein, which is stable under recommended storage conditions for at least six months.

In an embodiment, the process produces a lyophilized pharmaceutical composition containing a protein, which is stable under recommended storage conditions for at least 18 months.

In an embodiment, the process produces a lyophilized pharmaceutical composition containing a protein, which has a purity of 99.0% or more after storage for six months at 2-8° C.

In an embodiment, the process produces a lyophilized pharmaceutical composition containing a protein, which has a purity of 96.0% or more after storage for six months at 25° C.

In an embodiment, the process produces a lyophilized pharmaceutical composition containing a protein, which has a purity of 89.0% or more after storage for six months at 40° C.

In an embodiment, the process produces a lyophilized pharmaceutical composition containing a protein, which has a 9.6% or less loss in purity after storage for six months.

The present invention further provides a product produced by the process.

In an embodiment, the product reconstitutes in water for injection within 15 minutes.

In an embodiment, the product reconstitutes in water for injection within 7, 8, 9, 10, 11, 12, 13 or 14 minutes.

In an embodiment, the product reconstitutes in water for injection within 6 minutes.

In an embodiment, the product reconstitutes to a protein concentration from 2 to 250 mg/ml.

In an embodiment, the product reconstitutes to a protein concentration from 65 to 250 mg/ml. In an embodiment, the product reconstitutes to a protein concentration from 80 to 120 mg/ml.

In an embodiment, the product reconstitutes to a protein concentration from 100 to 110 mg/ml.

The present invention further provides a process for producing an injectable pharmaceutical composition, comprising obtaining an amount of the lyophilized pharmaceutical composition comprising a protein produced by the process, and reconstituting the lyophilized pharmaceutical composition with water for injection within 15 minutes, thereby producing an injectable pharmaceutical composition.

The present invention further provides a method of treating a patient with a therapeutic protein composition, comprising obtaining an amount of the lyophilized pharmaceutical composition comprising a protein produced, reconstituting the lyophilized pharmaceutical composition with water for injection within 15 minutes to form a reconstituted solution, and administering the reconstituted solution to the patient, thereby treating the patient.

The present invention further provides a process for producing an injectable pharmaceutical composition, comprising obtaining an amount of the lyophilized pharmaceutical composition comprising a protein produced by the process, and reconstituting the lyophilized pharmaceutical composition with water for injecting within 6 minutes, thereby producing an injectable pharmaceutical composition.

The present invention further provides a method of treating a patient with a therapeutic protein composition, comprising a protein produced, reconstituting the lyophilized pharmaceutical composition with water for injection within 6 minutes to form a reconstituted solution, and administering the reconstituted solution to the patient, thereby treating the patient.

In an embodiment, the osmolality of the reconstituted solution is from 250 to 350 mOsm/kg. In an embodiment, the osmolality of the reconstituted solution is from 275 to 325 mOsm/kg. In an embodiment, the osmolality of the reconstituted solution is 300 mOsm/kg.

In an embodiment, the reconstituted solution has a pH of 6.9-7.5. In an embodiment, the reconstituted solution has a pH of 7.1-7.3. In an embodiment, the reconstituted solution has a pH of 7.2.

The present invention further provides a sealed package comprising the lyophilized pharmaceutical composition.

In an embodiment, the sealed package comprises 80-120 mg of protein. In an embodiment, the sealed package comprises 100-110 mg of protein.

In an embodiment, the pharmaceutical composition is stable under recommended storage conditions for at least 6-36 months. In an embodiment, the pharmaceutical composition is stable under recommended storage conditions for at least 6 months. In an embodiment, the pharmaceutical composition is stable under recommended storage conditions for at least 9, 12, 18, 24, 30, or 36 months. In a specific embodiment, the pharmaceutical composition meets or exceeds 1, 2, 3, 4, 5 or more of the stability parameters set forth in Table 17. In a specific embodiment, the pharmaceutical composition meets or exceeds 1, 2, 3, 4, 5 or more of the stability parameters set forth in Table 18. In a specific embodiment, the pharmaceutical composition meets or exceeds 1, 2, 3, 4, 5 or more of the stability parameters set forth in Table 19.

In an embodiment, the container is a vial.

In an embodiment, the vial is made of glass. In an embodiment, the vial is made of USP Type 1 glass. In an embodiment, the container is made of flint glass.

In an embodiment, the vial is closed by a stopper. In an embodiment, the stopper is sealed by an aluminum seal. In an embodiment, the stopper has a FLUROTEC™ coating.

In an embodiment, the volume of the vial is from 1.5 to 5 ml. In an embodiment, the volume of the vial is 3 ml.

In an embodiment, the sealing comprises inserting a stopper. In an embodiment, the stopper is elastomeric. In an embodiment, the stopper comprises rubber. In an embodiment, the stopper comprises butyl rubber. In an embodiment, the stopper is halogenated. In an embodiment, the stopper comprises chlorobutyl rubber. In an embodiment, the stopper is coated with a coating. In an embodiment, the coating is FLUROTEC™.

In an embodiment, the protein is a fusion protein. In an embodiment, the fusion protein is a fusion of human serum albumin and a therapeutic protein. In an embodiment, the therapeutic protein is one of: Interferon alpha (Interferon alfa-2b; Interferon alfa-2a; recombinant; Interferon alfa-nl; Interferon alfan3; Peginterferon alpha-2b; Ribavirin and interferon alfa-2b; Interferon alfacon-l; interferon consensus; YM 643; CIFN; interferonalpha consensus; recombinant methionyl consensus interferon; recombinant consensus interferon; CGP 35269; RO 253036; RO 258310; Intron A; Pegintron; Oif; Omniferon; Pegomniferon; Veldona; Pegrebetron; Roferon A; Wellferon; Alferon N/Ldo; Rebetron; Altemol; Viraferonpeg; Pegasys; Viraferon; Virafon; Ampligen; Infergen; Infarex; Oragen) Atrial natriuretic peptide (ANP; atrial natriuretic factor; ANF) B-type natriuretic peptide (BNP, brain natriuretic peptide) Long-acting natriuretic peptide (LANP; proANP(31-67)); Vessel Dialator (VDP; proANP-(79-98)); Kaliuretic Peptide (KUP; proANP-(99-126)); C-type Natriuretic Peptide (CNP); Dendroaspis natriuretic peptide (DNP); Beta defensin-2 (beta defensin 4; SAP1; DEFB2; HBD-2; DEFB-2; DEFB102; skin-antimicrobial peptide 1); Human chemokine HCC-1 (ckBeta-1; CKB-1; HWFBD); Fractalkine (neurotactin; chemokine CX3C); Oxyntomodulin; Killer Toxin; Killer Toxin Peptide (KP); TIMP-4 (Tissue Inhibitor of Metalloprotease); PYY (Peptide YY, including PYY 3-36 (amino acid residues 31-64 of full length PYY, amino acid residues 3-36 of mature PYY) and also including PYY(3-36) (G9R); Adrenomedullin; Ghrelin; Calcitonin gene-related peptide (CGRP); Insulin-like growth factor-1 (Mecasermin; Somazon; IGF-1; IGF-1 complex; CEP 151; CGP 35126; FK 780; Mecar; RHIGF-I; Somatomedin-1; Somatomedin-C; Somatokine; Myotrophin; IGEF; DepoIGF-1); Neuraminidase (Influenza A virus (A/Goose/Guangdong/1/96 (H5N1)); Hemagglutinin [Influenza A virus (A/HongKong/213/03 (HK213:H5N1))]; Butyryl-cholinesterase (BchE, Serum Cholinesterase, pseudo-cholinesterase El (CHE1)); Endothelin (ET-1; GenbankAccession No. NP_{—}001946); Mechano Growth Factor (MGF; IGF-IEc; Genbank Accession No. P05019). In an embodiment, the fusion protein is a fusion of human serum albumin and butyryl-cholinesterase. In an embodiment, the fusion protein is Composition 1. In an embodiment, the fusion protein is a fusion of human serum albumin and human growth hormone. Examples of such proteins which may be used in embodiments of the invention are disclosed in U.S. Patent Application Publication Nos. US 2011/0002888 and US 2009/0029914, and U.S. Pat. Nos. 7,569,384 and 7,482,013, each of which is hereby incorporated by reference. In an embodiment, the fusion protein is Composition 2. In an embodiment, the fusion protein is Composition 3. In an embodiment, the fusion protein is Composition 4. In an embodiment, the fusion protein is Composition 5.

In an embodiment, the protein is a therapeutic protein. In an embodiment, the protein is an antibody. In an embodiment, the protein is not an antibody.

In an embodiment, the protein is one of: Insulin; Humulin; Novolin; Insulin human inhalation; Exubera; Insulin aspart; Novolog (aspart); Insulin glulisine; Apidra (glulisine); Insulin lispro; Humalog (lispro); Isophane insulin; NPH; Insulin detemir; Levemir (detemir); Insulin glargine; Lantus (glargine); Insulin zinc extended; Lente; Ultralente; Pramlintide acetate; Symlin; Growth hormone (GH); somatotropin; genotropin; humatrope; norditropin; NorIVitropin; Nutropin; Omnitrope; Protropin; Siazen; Serostim; Valtropin; Mecasermin; Increlex; Mecasermin rinfabate; IPlex; Factor VIII; Bioclate; Helixate; Kogenate; Recominate; ReFacto; Factor IX; Benefix; Antithromin III (AT-III); Thrombate III; Protein C concentrate; Ceprotin; β-Glucocerebrosidase; Cerezyme; β-Glucocerebrosidase; Ceredase (purified from pooled human placenta); Alglucosidase-α; Myozyme; Laronidase (α-l-iduronidase); Aldurazyme; Idursulphase (Iduronate-2-sulphatase); Elaprase; Galsulphase; Naglazyme; Agalsidase-β (human α-galactosidase A); Fabrazyme; α-1-Proteinase inhibitor; Aralast; Prolastin; Lactase; Lactaid; Pancreatic enzymes (lipase, amylase, protease); Arco-Lase, Cotazym, Creon, Donnazyme, Pancrease, Viokase, Zymase, Adenosine deaminase (pegademase bovine, PEG-ADA); Adagen; Pooled immunoglobulins; Octagam; Human albumin; Albumarc; Albumin; Albuminar; AlbuRx; Albutein; Flexbumin; Buminate; Plasbumin; Erythropoietin; Epoetin-α; Epogen; Procrit; Darbepoetin-α; Aranesp; Filgrastim (granulocyte colony stimulating factor; G-CS F); Neupogen; Pegfilgrastim (Peg-G-CSF); Neulasta; Sargramostim (granulocytemacrophage colony stimulating factor; GM-CS F); Leukine; Oprelvekin (interleukin11; IL11); Neumega; Human follicle-stimulating hormone (FSH); Gonal-F; Follistim; Human chorionic gonadotropin (HCG); Ovidrel; Luveris; Type 1 alpha-interferon; interferon alfacon 1; consensus interferon; Infergen; Interferon-α2a (IFNα2a); Roferon-A; PegInterferon-α2a; Pegasys; Interferon-α2b (IFNα2b); Intron A; PegInterferon-α2b; Peg-Intron; Interferon-αn3 (IFNαn3); Alferon N; Interferon-β1a (rIFN-β); Avonex; Rebif; Interferon-β1b (rIFN-β); Betaseron; Interferon-γ1b (IFNγ); Actimmune; Aldesleukin (interleukin 2 (IL2); epidermal thymocyte activating factor; ETAF); Proleukin; Alteplase (tissue plasminogen activator; tPA); Activase; Reteplase (deletion mutein of tPA); Retavase; Tenecteplase; TNKase; Urokinase; Abbokinase; Factor VIIa; NovoSeven; Drotrecogin-α (activated protein C); Xigris; Salmon calcitonin; Fortical; Miacalcin; Teriparatide (human parathyroid hormone residues 1-34); Forteo; Exenatide; Byetta; Octreotide; Sandostatin; Dibotermin-α (recombinant human bone morphogenic protein 2; rhBMP2); Infuse; Recombinant human bone morphogenic protein 7 (rhBMP7); Osteogenic protein 1; Histrelin acetate (gonadotropin releasing hormone; GnRH); Supprelin LA; Vantas; Palifermin (keratinocyte growth factor KGF); kepivance; Becaplermin (platelet-derived growth factor; PDGF); Regranex; Trypsin; Granulex; Nesiritide; Natrecor; Botulinum toxin type A; Botox; Botulinum toxin type B; Myoblock; Collagenase; Santyl; Human deoxy-ribonuclease I; dornase-α; pulmozyme; Hyaluronidase (bovine, ovine); Amphadase (bovine); hydase (bovine); Vitrase (ovine); Hyaluronidase (recombinant human); hylenex; Papain; accuzyme; panafil; L-asparaginase; ELSPAR; Peg-asparaginase; Oncaspar; Rasburicase; Elitek; Lepirudin; Refludan; Bivalirudin; Angiomax; Streptokinase; Streptase; Anistreplase (anisoylated plasminogen streptokinase activator complex; APSAC); Eminase; Bevacizumab; Avastin; Cetuximab; Erbitux; Panitumumab; Vectibix; Alemtuzumab; Campath; Rituximab; Rituxan; Trastuzumab; Herceptin; Abatacept; Orencia; Anakinra; Antril; Kineret; Abalimumab; Humira; Etanercept; Enbrel; Infliximab; Remicade; Alefacept; Amevive; Natalizumab; Tysabri; Eculizumab; Soliris; Antithymocyte globulin (rabbit); Thymoglobulin; Basiliximab; Simulect; Daclizumab; Zenapax; Muromonab-CD3; Orthoclone; OKT3; Omalizumab; Xolair; Palivizumab; Synagis; Enfuvirtide; Fuzeon; Abciximab; ReoPro; Pegvisomant; Somavert; Crotalidae polyvalent immune Fab (ovine); Crofab; Digoxin immune serum Fab (ovine); Digifab; Ranibizumab; Lucentis; Denileukin; Diftitox; Ontak; Ibritumomab; Tiuxetan; Zevalin; Gemtuzumab; Ozogamicin; Mylotarg; Tositumomab and I-tositumomab; Bexxar; Bexxar 1-131; Hepatitis B surface antigen (HBsAg); Engerix; Recombivax HB; HPV vaccine; Gardasil; OspA; LYMErix; Anti-Rhesus (Rh) immunoglobulin G; Rhophylac; Recombinant purified protein derivative (DPPD); Glucagon; GlucaGen; Growth hormone releasing hormone (GHRH); Geref; Secretin; ChiRhoStim (human peptide), SecreFlo (porcine peptide); Thyroid stimulating hormone (TSH); thyrotropin; Capromab pendetide; ProstaScint; Indium-111-octreotide; OctreoScan; Satumomab pendetide; OncoScint; Arcitumomab; CEA-scan; Nofetumomab; Verluma; Apcitide; Acutect; Imciromab pentetate; Myoscint; Technetium fanolesomab; NeutroSpec; HIV antigens; Enzyme immunoassay; OraQuick; Uni-Gold; Hepatitis C antigens; Recombinant immunoblot assay (RI BA). Examples of proteins which may be used in this invention are disclosed in Leader et al. 2008, which is hereby incorporated by reference.

For the foregoing embodiments, each embodiment disclosed herein is contemplated as being applicable to each of the other disclosed embodiment.

All combinations and sub-combinations of each of the various elements of the methods and embodiments described herein are envisaged and are within the scope of the invention.

This invention will be better understood by reference to the Examples which follow, which are set forth to aid in an understanding of the subject matter but are not intended to, and should not be construed to, limit in any way the claims which follow thereafter.

### EXAMPLES

### Example 1

### Experimental Determination of Novel Formulation

Composition 1, a recombinant protein composed of the mature form of recombinant human serum albumin (rHSA) fused at its amino terminus to the carboxy-terminus of a mutated human butyrylcholinesterase (BChE), was used to develop a novel lyophilization process and a suitable formulation. U.S. Provisional Application No. 61/752,740, filed Jan. 15, 2013, is hereby incorporated by reference into this application.

### Pre-Formulation Studies

### Ionic Strength Effects

### Sodium Chloride Spiking

Ionic strength effects were evaluated with Composition 1 (50 mg/mL in PMTT (which comprises 10 mM phosphate, 200 mM mannitol, 60 mM trehalose and 0.01% PS80, at pH 7.2)) at six target sodium chloride concentrations (5 mM, 10 mM, 20 mM, 50 mM, 80 mM, 120 mM).

Vials of each sample were incubated at 25° C. for 5 days. Samples were removed from incubation after 5 days. The samples were compared to the 0 day and 0 mM sodium chloride controls by visual inspection and SE-HPLC.

The results suggest that increased concentrations of sodium chloride reduce purity loss. At or above 6 mS/cm, there is no significant change in SE-HPLC purity (Table 1). All tested samples were clear, pale yellow, and essentially free from foreign particulate matter.

TABLE 1
Ionic Strength Effects Measured by Sodium Chloride Spiking.

| NaCl | | SE-HPLC | SE-HPLC |
|---|---|---|---|
| (mM) | Day | purity (%) | purity loss (%) |
| 0 | 0 | 99.8 | NA |
| | 5 | 95.1 | −4.7 |
| 5 | 0 | 99.8 | NA |
| | 5 | 95.9 | −3.9 |
| 10 | 0 | 99.8 | NA |
| | 5 | 96.0 | −3.8 |
| 20 | 0 | 99.9 | NA |
| | 5 | 96.3 | −3.6 |
| 50 | 0 | 99.8 | NA |
| | 5 | 99.7 | −0.1 |
| 80 | 0 | 99.8 | NA |
| | 5 | 99.7 | −0.1 |
| 120 | 0 | 99.8 | NA |
| | 5 | 99.8 | 0.0 |

Buffer controls containing 5 mM, 10 mM, 20 mM, 50 mM, 80 mM, and 120 mM sodium chloride were measured for conductivity. Buffer controls containing 10 mM, 20 mM, 30 mM, 40 mM, 50 mM, and 60 mM phosphate were measured for conductivity.

When the concentration of phosphate is 50 mM, the conductivity of the solution is ≧6 mS/cm (Table 2). Therefore, phosphate can be used to replace NaCl while maintaining the ionic strength.

TABLE 2
Sodium Chloride and Sodium Phosphate
Buffer Conductivity Comparison.

| NaCl | Conductivity | Phosphate | Conductivity |
|---|---|---|---|
| (mM) | (mS/cm) | (mM) | (mS/cm) |
| 0 | 1.29 | 10 | 1.21 |
| 5 | 1.78 | 20 | 2.93 |
| 10 | 2.30 | 30 | 4.52 |
| 20 | 3.30 | 40 | 6.06 |
| 50 | 6.32 | 50 | 7.46 |
| 80 | 9.21 | 60 | 8.71 |
| 120 | 12.62 | | |

### Phosphate Spiking

Ionic strength effects were evaluated with Composition 1 (100 mg/mL in 200 mM mannitol, 60 mM trehalose, 0.03% PS80, pH 7.2) at six target phosphate concentrations (10 mM, 20 mM, 30 mM, 40 mM, 50 mM, 60 mM).

Vials of each sample were incubated at 25° C. for 5 days. Samples were removed from incubation after 3 and 5 days. The samples were compared to the 0 day controls by visual inspection and SE-HPLC. Buffer controls were measured for conductivity.

The results show that increasing buffer conductivity decreases SE-HPLC purity loss. At a conductivity of approximately 4.5 mS/cm or higher (˜≧30 mM sodium phosphate), there is no significant SE-HPLC purity loss after 5 days at 25° C. (, Table 3). All tested samples were clear, pale yellow, and essentially free from foreign particulate matter. Therefore, increasing ionic strength could prevent the protein from forming aggregates.

TABLE 3
Sodium Phosphate Spiking Data.

| Phosphate | | SE-HPLC | SE-HPLC |
|---|---|---|---|
| (mM) | Day | purity (%) | purity loss (%) |
| 10 | 0 | 99.6 | NA |
| | 3 | 94.6 | −5.1 |
| | 5 | 92.4 | −7.3 |
| 20 | 0 | 99.7 | NA |
| | 3 | 98.0 | −1.6 |
| | 5 | 97.3 | −2.4 |
| 30 | 0 | 99.7 | NA |
| | 3 | 99.0 | −0.7 |
| | 5 | 98.7 | −1.0 |
| 40 | 0 | 99.7 | NA |
| | 3 | 99.4 | −0.3 |
| | 5 | 99.2 | −0.4 |
| 50 | 0 | 99.6 | NA |
| | 3 | 99.5 | −0.2 |
| | 5 | 99.4 | −0.3 |
| 60 | 0 | 99.7 | NA |
| | 3 | 99.6 | −0.1 |
| | 5 | 99.5 | −0.2 |

### Polysorbate 80 Effects

The effects of PS80 were evaluated with Composition 1 (100 mg/mL in 10 mM phosphate, 200 mM mannitol, 60 mM trehalose, pH 7.2) at four target PS80 concentrations (0.01%, 0.05%, 0.1%, and 0.2%). The samples were incubated at 2-8° C. and 25° C. for 1, 2 and 3 days. Samples were compared to the 0 point and the PS80-free controls by visual inspection and SE-HPLC. Osmolality was measured for the 0 points.

There was no change in purity for samples incubated at 2-8° C. (Table 4). Samples at 100 mg/ml in PMTT incubated at 25° C. showed 5-6% purity loss, but with no significant differences across the PS80 concentrations (Table 4). There was no change in appearance across all PS80 concentrations, temperatures and time points, with the reconstituted solution always a clear pale yellow liquid essentially free from foreign particulate matter. There was no change in osmolality (Table 5). Since there was no significant difference, 0.03% PS80, considered an acceptable middle point, was selected. This data also demonstrated that PMTT was not a suitable formulation for a higher dose of concentrated product.

TABLE 4
PS80 Spiking Purity Data

| | | SEC Purity (%) | | | | | SEC Purity Loss (%) | | | | |
|---|---|---|---|---|---|---|---|---|---|---|---|
| | | PS80 concentration (%) | | | | | PS80 concentration (%) | | | | |
| Temperature | Day | 0 | 0.01 | 0.05 | 0.1 | 0.2 | 0 | 0.01 | 0.05 | 0.1 | 0.2 |
| 2-8° C. | 0 | 99.7 | 99.7 | 99.8 | 99.8 | 99.7 | NA | NA | NA | NA | NA |
| | 1 | 99.7 | 99.7 | 99.7 | 99.7 | 99.7 | 0.0 | 0.0 | −0.1 | 0.0 | 0.0 |
| | 2 | 99.8 | 99.7 | 99.7 | 99.7 | 99.7 | 0.0 | 0.0 | −0.1 | 0.0 | 0.0 |
| | 3 | 99.7 | 99.7 | 99.7 | 99.7 | 99.7 | 0.0 | 0.0 | 0.0 | −0.1 | 0.0 |
| 25° C. | 0 | 99.7 | 99.7 | 99.8 | 99.8 | 99.7 | NA | NA | NA | NA | NA |
| | 1 | 97.5 | 97.5 | 97.6 | 97.6 | 97.6 | −2.2 | −2.2 | −2.2 | −2.1 | −2.1 |
| | 2 | 95.5 | 95.5 | 95.6 | 95.8 | 95.8 | −4.2 | −4.3 | −4.1 | −4.0 | −3.9 |
| | 3 | 94.0 | 94.1 | 94.1 | 94.3 | 94.3 | −5.7 | −5.6 | −5.7 | −5.5 | −5.5 |

TABLE 5
PS80 Spiking Osmolality Data
Osmolality Average (mOsm/kg)
PS80 concentration (%)

| 0 | 0.01 | 0.05 | 0.1 | 0.2 |
|---|---|---|---|---|
| 340 | 341 | 345 | 341 | 347 |

### Buffer Composition

Formulation buffers containing varying concentrations of phosphate (40 mM, 50 mM and 60 mM), mannitol (60-200 mM), trehalose (18-60 mM) and 0.03% PS80 were made by combining varying amounts of 500 mM phosphate (pH 7.2) stock solution, 500 mM mannitol stock solution and 200 mM trehalose stock solution, while keeping the ratio of trehalose to mannitol the same as PMTT. The osmolality of each buffer was tested and compared to the osmolality of PMTT (Table 6).

TABLE 6
Phosphate Buffer Combinations

| PS80 | Phosphate | Mannitol | Trehalose | Osm Average |
|---|---|---|---|---|
| (%) | (mM) | (mM) | (mM) | (mOsm/kg) |
| 0.01 | 10 | 200 | 60 | 309 |
| 0.03 | 40 | 200 | 60 | 417 |
| 0.03 | 40 | 160 | 48 | 360 |
| 0.03 | 40 | 154 | 46 | 352 |
| 0.03 | 40 | 150 | 45 | 342 |
| 0.03 | 40 | 146 | 44 | 341 |
| 0.03 | 40 | 140 | 42 | 355 |
| 0.03 | 40 | 120 | 36 | 308 |
| 0.03 | 40 | 114 | 34 | 299 |
| 0.03 | 40 | 110 | 33 | 294 |
| 0.03 | 40 | 106 | 32 | 289 |
| 0.03 | 40 | 100 | 30 | 281 |
| 0.03 | 50 | 200 | 60 | 454 |
| 0.03 | 50 | 150 | 45 | 381 |
| 0.03 | 50 | 146 | 44 | 378 |
| 0.03 | 50 | 140 | 42 | 367 |
| 0.03 | 50 | 134 | 40 | 365 |
| 0.03 | 50 | 130 | 39 | 356 |
| 0.03 | 50 | 100 | 30 | 315 |
| 0.03 | 50 | 94 | 28 | 306 |
| 0.03 | 50 | 90 | 27 | 301 |
| 0.03 | 50 | 86 | 26 | 297 |
| 0.03 | 50 | 80 | 24 | 285 |
| 0.03 | 60 | 200 | 60 | 484 |
| 0.03 | 60 | 134 | 40 | 406 |
| 0.03 | 60 | 130 | 39 | 384 |
| 0.03 | 60 | 126 | 38 | 382 |
| 0.03 | 60 | 120 | 36 | 372 |
| 0.03 | 60 | 114 | 34 | 363 |
| 0.03 | 60 | 110 | 33 | 361 |
| 0.03 | 60 | 80 | 24 | 318 |
| 0.03 | 60 | 74 | 22 | 313 |
| 0.03 | 60 | 70 | 21 | 308 |
| 0.03 | 60 | 66 | 20 | 303 |
| 0.03 | 60 | 60 | 18 | 297 |

Buffers with osmolality approximately equal to 300 mOsm/kg were made, and conductivity and osmolality were measured for the buffers and for Composition 1 (100 mg/mL in PMTT) (Table 7).

TABLE 7
Proto-formulation Buffer Measurements
(Bolded lines indicate P50MTT and P60MTT)

| | | | | | | Osm | Conductivity |
|---|---|---|---|---|---|---|---|
| | Phosphate | Mannitol | Trehalose | PS80 | | Average | Average |
| Sample | (mM) | (mM) | (mM) | (%) | pH | (mOsm/kg) | (mS/cm) |
| Buffer | 10 | 200 | 60 | 0.01 | 7.23 | 302 | 1.29 |
| Buffer | 10 | 200 | 60 | 0.01 | NT | 311 | NT |
| Composition 1 | 10 | 200 | 60 | 0.01 | NT | 338 | NT |
| (100 mg/mL) | | | | | | | |
| Buffer | 40 | 114 | 34 | 0.03 | 7.18 | 243 | NT |
| Buffer | 40 | 132 | 40 | 0.03 | 7.24 | 267 | 4.55 |
| Buffer | 40 | 146 | 44 | 0.03 | 7.20 | 289 | 4.46 |
| Buffer | 40 | 150 | 45 | 0.03 | 7.19 | 293 | 4.46 |
| Buffer | 50 | 90 | 27 | 0.03 | 7.20 | 232 | NT |
| Buffer | 50 | 94 | 28 | 0.03 | 7.20 | 235 | NT |
| Buffer | 50 | 115 | 35 | 0.03 | 7.18 | 267 | 5.54 |
| Buffer | 50 | 116 | 35 | 0.03 | 7.18 | 272 | 5.61 |
| Buffer | 50 | 134 | 40 | 0.03 | 7.25 | 294 | 5.52 |
| Buffer | 50 | 140 | 42 | 0.03 | 7.16 | 302 | 5.23 |
| Buffer | 60 | 60 | 18 | 0.03 | 7.20 | 211 | NT |
| Buffer | 60 | 66 | 20 | 0.03 | 7.18 | 219 | NT |
| Buffer | 60 | 100 | 30 | 0.03 | 7.21 | 268 | 6.59 |
| Buffer | 60 | 104 | 31 | 0.03 | 7.16 | 275 | 6.56 |
| Buffer | 60 | 120 | 36 | 0.03 | 7.21 | 290 | 6.31 |
| Buffer | 60 | 126 | 38 | 0.03 | 7.18 | 301 | 6.42 |

From measuring Composition 1 (100 mg/mL in PMTT) and PMTT alone, it was calculated that Composition 1 at 100 mg/mL contributes approximately 31.5 mOsm/kg to osmolality. Targeting an osmolality of 300 mOsm/kg, two formulations were selected: P50MTT (267 mOsm/kg), and P60MTT (268 mOsm/kg). P50MTT comprises 50 mM sodium phosphate, 115 mM mannitol, 35 mM trehalose and 0.03% PS80, at pH 7.2, while P60MTT comprises 60 mM phosphate, 100 mM mannitol, and 30 mM trehalose and 0.03% PS80, at pH 7.2.

Measurements were performed for Composition 1 (100 mg/mL) in the new P50MTT and P60MTT formulations (Table 8).

TABLE 8
P50MTT and P60MTT Measurements

| | | | Osmolality | | |
|---|---|---|---|---|---|
| | | Osmolality | sample | Density | |
| Formula- | Conductivity | buffer | (100 mg/ml) | buffer | pH |
| tion | (mS/cm) | (mOsm/kg) | (mOsm/kg) | (g/cm³) | buffer |
| P50MTT | 5.75 | 274 | 307 | 1.015 | 7.09 |
| P60MTT | 6.77 | 272 | 306 | 1.015 | 7.09 |

### Pre-Formulation Conclusions

The pre-formulation studies were executed to determine potential formulation candidates for the lyophilization formulation of the concentrated product. Previous studies showed that Composition 1 was affected by concentration dependent aggregation, suggesting that aggregation is a major degradation pathway.

In response, the ionic strength study was conducted to determine if increasing the ionic strength of the formulation buffer would have an effect on reducing aggregation. The results of the study demonstrate that there is a significant ionic strength effect, and in the higher ionic strength formulation there was a significant reduction in dose dependent aggregation at a protein concentration of 100 mg/ml.

The results of the PS80 spiking study show no difference between PS80 concentrations. Therefore, 0.03% PS80, which is within the acceptable range, was selected for the formulations.

Mannitol and trehalose concentrations in the candidate formulations were modified to target an osmolality of 300 mOsm/kg, while maintaining the ratio between mannitol and trehalose as established during development of the previous PMTT formulation. Two proto-formulations, P50MTT and P60MTT, were selected for additional studies.

### Proto-Formulation Evaluation

### Freeze-Thaw Effects

The effects of repeated freezing and thawing were evaluated with Composition 1 (101.6 mg/mL in P50MTT and 100.8 mg/mL in P60MTT). Samples were frozen for 2-16 hours at ≦−65° C. and then thawed for 3 hours at room temperature. Samples were collected after 1, 2, 4, 6 and 10 complete cycles of freezing and thawing. Samples were compared to the 0 point by visual inspection and SE-HPLC. Select samples were also tested by SDS-PAGE and potency analysis.

The results show no change in SE-HPLC purity after 10 cycles of freeze and thaw on Composition 1 in both P50MTT and P60MTT. The SDS-PAGE results support the results of SE-HPLC. All tested samples were clear, pale yellow, and essentially free from foreign particulate matter. There was no significant change in potency (Table 9).

TABLE 9
Freeze-Thaw Effects on Composition 1

| | | SEC % | SE-HPLC | |
|---|---|---|---|---|
| | | Purity | Purity | Potency |
| Formulation | Cycle | Average | Change (%) | (%) |
| P50MTT | 0 | 99.3 | NA | 166 |
| | 1 | 99.3 | 0.0 | NT |
| | 2 | 99.4 | 0.1 | NT |
| | 4 | 99.4 | 0.1 | NT |
| | 6 | 99.4 | 0.1 | NT |
| | 10 | 99.4 | 0.1 | 137 |
| P60MTT | 0 | 99.5 | NA | 145 |
| | 1 | 99.4 | 0.0 | NT |
| | 2 | 99.4 | 0.0 | NT |
| | 4 | 99.4 | −0.1 | NT |
| | 6 | 99.4 | 0.0 | NT |
| | 10 | 99.4 | −0.1 | 138 |

### Shaking Effects

The effects of shaking-induced aggregation were evaluated with Composition 1 (101.6 mg/mL in P50MTT and 100.8 mg/mL in P60MTT). Samples were shaken horizontally at 150 rpm. Samples were incubated at 2-8° C. and 25° C. from 0 to 24 hours. Samples were compared to the 0 point by visual inspection, SE-HPLC and HI-HPLC.

The results show no change in SE-HPLC purity or HI-HPLC purity for Composition 1 in both P50MTT and P60MTT. All tested samples were clear, pale yellow, and essentially free from foreign particulate matter. This suggests that Composition 1 is not sensitive to shaking induced aggregation (Table 10).

TABLE 10
Shaking Effects on Composition 1.

| | | | | SE- | | |
|---|---|---|---|---|---|---|
| | | | SE- | HPLC | HI- | HI-HPLC |
| | | | HPLC | Purity | HPLC | Purity |
| | | | Purity | Change | Purity | Change |
| Formulation | Temp | Hrs | (%) | (%) | (%) | (%) |
| P50MTT | 2-8° C. | 0 | 99.5 | NA | 91.5 | NA |
| | | 1 | 99.5 | 0.0 | NT | NT |
| | | 3 | 99.6 | 0.1 | 91.6 | 0.1 |
| | | 6 | 99.5 | 0.0 | NT | NT |
| | | 12 | 99.5 | 0.0 | 91.6 | 0.1 |
| | | 24 | 99.5 | 0.0 | 91.6 | 0.1 |
| | 22° C. | 0 | 99.5 | NA | 91.5 | NA |
| | | 1 | 99.4 | 0.0 | NT | NT |
| | | 3 | 99.5 | 0.0 | 91.7 | 0.2 |
| | | 6 | 99.5 | 0.0 | NT | NT |
| | | 12 | 99.4 | 0.0 | 91.6 | 0.1 |
| | | 24 | 99.4 | −0.1 | 91.6 | 0.1 |
| P60MTT | 2-8° C. | 0 | 99.5 | NA | 91.7 | NA |
| | | 1 | 99.5 | 0.0 | NT | NT |
| | | 3 | 99.5 | 0.0 | 91.7 | 0.0 |
| | | 6 | 99.5 | 0.0 | NT | NT |
| | | 12 | 99.5 | 0.0 | 91.6 | −0.1 |
| | | 24 | 99.5 | 0.0 | 91.7 | 0.0 |
| | 22° C. | 0 | 99.5 | NA | 91.7 | NA |
| | | 1 | 99.505 | 0.0 | NT | NT |
| | | 3 | 99.525 | 0.0 | 91.6 | −0.1 |
| | | 6 | 99.505 | 0.0 | NT | NT |
| | | 12 | 99.5 | 0.0 | 91.6 | −0.1 |
| | | 24 | 99.48 | 0.0 | 91.7 | 0.0 |

### Short-Term Liquid Stability

Composition 1 (101.6 mg/mL P50MTT and 100.8 mg/mL in P60MTT) was used for this study. Samples were incubated at 2-8° C. and 25° C. for 6 days. Samples were removed from incubation after 1, 3 and 6 days. Samples were compared to the 0 point by visual inspection, SE-HPLC and HI-HPLC. All tested samples were clear, pale yellow, and essentially free from foreign particulate matter. Select samples were also tested by SDS-PAGE and potency analysis (Table 11).

TABLE 11
Short Term Liquid Stability Results

| | | | SE-HPLC | SE-HPLC | HI-HPLC | HI-HPLC | |
|---|---|---|---|---|---|---|---|
| Formulation | | | Purity | Purity | Purity | Purity | Potency |
| (100 mg/mL) | Temp | Day | (%) | Loss (%) | (%) | Loss (%) | (%) |
| PMTT | 25° C. | 0 | 99.6 | NA | 88.9 | NA | NT |
| | | 5 | 95.1 | −4.5 | 81.7 | −7.2 | NT |
| P50MTT | 2-8° C. | 0 | 99.4 | NA | 92.0 | NA | 131 |
| | | 1 | 99.4 | 0.0 | NT | NT | NT |
| | | 3 | 99.4 | 0.0 | 91.9 | −0.1 | NT |
| | | 6 | 99.3 | 0.0 | 91.9 | −0.1 | 147 |
| | 25° C. | 0 | 99.4 | NA | 92.0 | NA | 131 |
| | | 1 | 99.3 | −0.1 | NT | NT | NT |
| | | 3 | 99.2 | −0.2 | 92.0 | 0.0 | NT |
| | | 6 | 99.0 | −0.3 | 92.0 | 0.0 | 120 |
| P60MTT | 2-8° C. | 0 | 99.4 | NA | 92.0 | NA | 154 |
| | | 1 | 99.4 | 0.0 | NT | NT | NT |
| | | 3 | 99.4 | 0.0 | 92.1 | 0.1 | NT |
| | | 6 | 99.4 | 0.0 | 92.1 | 0.1 | 129 |
| | 25° C. | 0 | 99.4 | NA | 92.0 | NA | 154 |
| | | 1 | 99.4 | 0.0 | NT | NT | NT |
| | | 3 | 99.2 | −0.2 | 92.1 | 0.1 | NT |
| | | 6 | 99.1 | −0.3 | 91.9 | −0.1 | 126 |

The results show that Composition 1 in both P50MTT and P60MTT had no change in SE-HPLC and HI-HPLC purity (after incubation in 2-8° C. and 25° C. for 6 days. This is a significant change from the prior formulation (PMTT), which had an approximate SE-HPLC purity loss of 4.5% and HI-HPLC purity loss of 7.2% after incubation at 25° C. after 5 days. The SDS-PAGE results support the results of SE-HPLC. There was no significant change in potency (Table 11).

### Proto-Formulation Conclusion

The results of the proto-formulation studies indicate that Composition 1 at 100 mg/mL is stable at 2-8° C. and 25° C. for up to 6 days in both P50MTT and P60MTT formulations. Composition 1 in P50MTT and in P60MTT was not sensitive to freeze-thaw or shaking effects.

Overall, there was no difference between the P50MTT and P60MTT formulations. Both could support the lyophilization process and would be potential formulation candidates for an initial lyophilization evaluation.

### Lyophilization Formulation Evaluation

### Initial Lyophilization Evaluation

An initial lyophilization cycle evaluation was carried out using Composition 1 (101.6 mg/mL in P50MTT and 100.8 mg/mL in P60MTT). The TBU lyophilization cycle is summarized in Table 12. Post-lyophilization tests include visual inspection pre- and post-reconstitution and residual moisture content analysis. 0-12 hour post-reconstitution samples were analyzed by SE-HPLC and HI-HPLC. Selected samples were also tested by potency analysis.

TABLE 12
TBU Lyophilization Cycle

| Step | Parameters |
|---|---|
| a | Set the shelf temperature to 5° C. and load the samples. |
| b | Hold at 5° C. for 2 hours. |
| c | Ramp to −45° C. over 2.8 hours (0.3° C./min). |
| d | Hold at −45° C. for 3 hours. |
| e | Ramp to −18° C. over 0.6 hour (0.8° C./min). |
| f | Hold at −18° C. for 5 hours. |
| g | Ramp to −45° C. over 1.5 hours (0.3° C./min). |
| h | Hold at −45° C. for 2 hours. |
| i | Control pressure at 100 mT. |
| j | Hold at −45° C. for 1 hour. |
| k | Increase shelf temp to −10° C. over 0.8 hour (0.6° C./min). |
| l | Hold at −10° C. for 36 hours. |
| m | Increase shelf temp to 25° C. over 0.8 hour (0.6° C./min). |
| n | Hold at 25° C. for 15 hours. |
| o | Restore the chamber to partial atmospheric pressure. |
| p | Stopper the product. |

The lyophilization products were pharmaceutically acceptable cakes (white to off-white in color and intact).

There was no change in SE-HPLC purity, HI-HPLC purity, and potency between pre- and post-lyophilization. The residual moisture content for both cakes was 0.1%.

### Lyophilization Cycle Evaluation

### Low Temperature Thermal Analysis

To characterize the physio-chemical behavior of Composition 1 (100 mg/mL in P50MTT) at low temperatures, low temperature thermal analysis was performed. The analysis consisted of electrical resistance measurements (using a Kaye Validator instrument), observations of freeze drying behavior using a freeze-drying microscope (FDM), and low temperature differential scanning calorimetry (LT-DSC).

The results of the analysis are summarized below:
Phase transition at −17° C.
A minimum temperature of −29° C. is required for complete solidification
Liquid-like movement occurs at −4° C.
Recommended temperature for primary drying at or below a range of −6° C. to −8° C.

The TBU lyophilization cycle conditions are summarized as follows:
Freezing and refreezing steps at −45° C.
Annealing step at −18° C.
Primary drying at −10° C.

This data supports that the TBU lyophilization cycle is appropriately designed and suitable for this product.

### TBU and Other Lyophilization Cycle Evaluations

Composition 1 (100 mg/mL in P50MTT) was lyophilized using the TBU cycle and used for the long term stability study.

Two randomly selected vials from the batch were analyzed by visual inspection and pre and post lyophilization analysis.

The results indicate that the TBU cycle produces pharmaceutically acceptable cakes that are white to off-white in color and intact.

Additional lyophilization cycle evaluation was carried out using Composition 1 (103 mg/mL in P50MTT). A total of 7 development lyophilization cycles, as well as the TBU cycle as a control, were completed with variations to the freezing, annealing, primary drying, and secondary drying steps. Upon completion of the lyophilization process, samples were analyzed by visual inspection, moisture content analysis, HI-HPLC and SE-HPLC.

The lyophilization cycle evaluation was carried out using Composition 1 (103 mg/mL in P50MTT). Visual inspection, residual moisture content measurement, SE-HPLC and HI-HPLC purity analysis were performed. See Table 13 for detailed information pertaining to the various lyophilization cycle parameters.

TABLE 13
Lyophilization Cycle Parameters Summary

| | | Freezing | Annealing | Refreeze | | Secondary | |
|---|---|---|---|---|---|---|---|
| | Shelf | Step | Step | Step | | drying | Total |
| | loading | (final temp, | (final temp, | (final temp, | Primary drying | (final temp, rate, | cycle |
| Lyo | temp | rate, hold | rate, hold | rate, hold | (final temp, rate, hold | hold duration, | time |
| Cycle | (C.) | duration) | duration) | duration) | duration, pressure) | pressure) | (hrs) |
| TBU | 5 | −45° C., | −18° C., | −45° C., | −45° C., 0.0° C./min, | 25° C., 0.8° C./min, | 70 |
| | | 0.3° C./min, | 0.8° C./min, | 0.3° C./min, | 1 hour, | 15 hour, | |
| | | 3 hours | 5 hours | 2 hours | −10° C., 0.8° C./min, | 100 mTorr | |
| | | | | | 36 hours, | | |
| | | | | | 100 mTorr | | |
| 1^{@} | 10 | −45° C., | — | — | −10° C., 0.2° C./min, | 25° C., 0.3° C./min, | 25 |
| | | 0.5° C./min, | | | 13 hours, | 4 hour, | |
| | | 2 hours | | | 150 mTorr | 150 mTorr | |
| 2^{@} | 10 | −35° C., | −17° C., | −40° C., | −10° C., 0.2° C./min, | 25° C., 0.3° C./min, | 24.5 |
| | | 0.6° C./min, | 0.3° C./min, | 0.4° C./min, | 10 hours, | 1 hour, | |
| | | 2 hours | 2 hours | 1 hour | 150 mTorr | 150 mTorr | |
| 3^{#} | 10 | −35° C., | −17° C., | −40° C., | −10° C., 0.2° C./min, | 25° C., 0.3° C./min, | 25.5 |
| | | 0.6° C./min, | 0.3° C./min, | 0.4° C./min, | 10 hours, | 2 hours, | |
| | | 2 hours | 2 hours | 1 hour | 300 mTorr | 300 mTorr | |
| 4^{#} | 10 | −35° C., | −15° C., | −40° C., | −30° C., 0.2° C./min, | 0° C., | 32 |
| | | 0.4° C./min, | 0.3° C./min, | 0.4° C./min, | 10 hours, | 0.5° C./min, | |
| | | 2 hours | 2 hours | 1 hour | −15° C., 0.1° C./min, | 4.5 hours, | |
| | | | | | 10 hours, | 100 mTorr | |
| | | | | | 100 mTorr | | |
| 5^{#} | 10 | −35° C., | −17° C., | −40° C., | −10° C., 0.2° C./min, | 25° C., 0.6° C./min, | 27.5 |
| | | 0.6° C./min, | 0.3° C./min, | 0.4° C./min, | 11 hours, | 1 hour, | |
| | | 2 hours | 4 hours | 2 hour | 500 mTorr | 500 mTorr | |
| 6^{#} | 10 | −35° C., | −15° C., | −40° C., | −10° C., 0.1° C./min, | 25° C., 0.3° C./min, | 32.5 |
| | | 0.3° C./min, | 0.3° C./min, | 0.2° C./min, | 11 hours, | 5 hour, | |
| | | 2 hours | 4 hours | 2 hour | 500 mTorr | 500 mTorr | |
| 7^{#} | −40 | −40° C., 4.25 | — | — | −10° C., 0.1° C./min, | 25° C., 0.6° C./min, | 25.25 |
| | | hours | | | 12 hours, | 5 hour, | |
| | | | | | 500 mTorr | 500 mTorr | |

The results of the lyophilization cycle evaluation further confirm that the TBU lyophilization cycle is more appropriate for Composition 1. The data suggests that the TBU cycle produces pharmaceutically acceptable cakes, with the lowest residual moisture (0.3%) compared to the other lyophilization cycles tested during the evaluation (Table 14).

TABLE 14
Lyophilization Cycle Evaluation Results Summary

| | | | | | Reconstituted | Composition 1 | |
|---|---|---|---|---|---|---|---|
| | General | | Moisture | | product | concentration/purity | |
| Lyo | Cycle | Cake | content | Reconstitution | appearance, | HI-HPLC\hat{ } | SE-HPLC\hat{ } |
| Cycle | Parameters | Appearance | (% w/w) | time (min)* | sample pH | (mg/mL, %) | (mg/mL, %) |
| TBU | Anneal at | White, intact | 0.3 | See Table 17 | No particulates | 98, 89.6 | 99, 99.5 |
| | −18° C. for 5 h, | cake, | | for TBU data | visible, color | | |
| | primary at | separation | | | same as starting | | |
| | −10° C. for | from vial side, | | | material, pH 7.10 | | |
| | 36 h, | slight top | | | | | |
| | 100 mT | edge cracking | | | | | |
| 1^{@} | Direct freeze | White, intact | 0.6 | 29.5 | No particulates | 108, 90.3 | 100, 99.4 |
| | to −45° C., | cake, | | | visible, color | | |
| | primary at | separation | | | same as starting | | |
| | −10° C. for | from vial side | | | material, | | |
| | 13 h, 150 mT | | | | pH 7.09 | | |
| 2^{@} | Anneal at | White, intact | 0.8 | 17.5 | No particulates | 109, 90.8 | 102, 99.4 |
| | −17° C. for 2 h, | cake, slight | | | visible, color | | |
| | primary at | separation | | | same as starting | | |
| | −10° C. for | from vial side, | | | material, | | |
| | 10 h, 150 mT | slight top | | | pH 7.06 | | |
| | | edge cracking | | | | | |
| 3^{#} | Anneal at | White, intact | 0.6 | 19.5 | No particulates | 118, 90.4 | 113, 99.5 |
| | −17° C. for 2 h, | cake, | | | visible, color | | |
| | primary at | separation | | | same as starting | | |
| | −10° C. for | from vial side | | | material, | | |
| | 10 h, 300 mT | | | | pH 7.08 | | |
| 4^{#} | Anneal at | White, intact | 0.6 | 24.0 | No particulates | 118, 90.4 | 114, 99.5 |
| | −15° C. for 2 h, | cake, | | | visible, color | | |
| | primary at | separation | | | same as starting | | |
| | −30° C. for | from vial side, | | | material, | | |
| | 10 h and | slight top | | | pH 7.09 | | |
| | −15° C. for | edge cracking | | | | | |
| | 10 h, 100 mT | | | | | | |
| 5^{#} | Anneal at | White, intact | 1.2 | 23.5 | No particulates | 106, 89.6 | 108, 99.4 |
| | −17° C. for 3 h, | cake, contact | | | visible, color | | |
| | primary at | with vial side, | | | same as starting | | |
| | −10° C. for | slight top | | | material, | | |
| | 11 h, 500 mT | edge cracking | | | pH 7.06 | | |
| 6^{#} | Anneal at | White, intact | 0.6 | 33.5 | No particulates | NT | NT |
| | −15° C. for 4 h, | cake, contact | | | visible, color | | |
| | 0.2° C./min | with vial side, | | | same as starting | | |
| | warming rate | slight top | | | material, | | |
| | to primary at | edge cracking | | | pH 7.09 | | |
| | −10° C., −10° C. | | | | | | |
| | for 11 h | | | | | | |
| | 500 mT | | | | | | |
| 7^{#} | Load | White, intact | NT | 14 | No particulates | 104, 91.0 | NT |
| | samples on | crystalline | | | visible, color | | |
| | pre-cooled | cake, | | | same as starting | | |
| | (−40° C.) helf, | separation | | | material, | | |
| | primary at | from vial side | | | pH 7.09 | | |
| | −10° C. for | | | | | | |
| | 10 h, 500 mT | | | | | | |

*Samples vials were reconstituted with 1 mL sWFI at ambient laboratory conditions. Samples were inverted 5X upon addition of sWFI and then incubated at ambient laboratory conditions without additional agitation until complete dissolution was observed.
^{@}1.0 mL fill volume.
^{#}1.1 mL fill volume.
\hat{ }Bulk TV-1380 purity 89.8%, 99.6% as determined by HIC and SEC-HPLC, respectively.

### Pre- and Post-Lyophilization Analysis

Composition 1 (100 mg/ml in P50MTT after reconstitution with 1.1 ml of WFI) 0 month was used for the pre- and post-lyophilization analysis. Time points were 0, 4, 8 and 12 hours. Visual inspection was performed prior to reconstitution. Reconstitution time was recorded. Post-reconstitution, samples were analyzed by visual inspection, pH, osmolality, concentration measurement, SE-HPLC, SDS-PAGE, potency analysis and free thiol content (Table 15).

TABLE 15
Pre and Post Reconstitution Summary

| | Results | | | |
|---|---|---|---|---|
| Attributes | 0 hr | 4 hr | 8 hr | 12 hr |
| Appearance (Visual | WC | WC | WC | WC |
| inspection - pre | | | | |
| reconstitution; cake) | | | | |
| Appearance (Visual | ≦4 min | ≦5 min | ≦5 min | ≦6 min |
| inspection - | | | | |
| reconstitution time) | | | | |
| Appearance (Visual | CYF | CYF | CYF | CYF |
| inspection - post | | | | |
| reconstitution) | | | | |
| pH | 7.2 | 7.2 | 7.2 | 7.1 |
| Osmolality (Freezing | 262 | 269 | 280 | 283 |
| point) (mOsm/kg) | | | | |
| Concentration | 93.1 | 96.8 | 98.7 | 102.3 |
| (A280) (mg/mL) | | | | |
| Purity (SDS-PAGE), | 100 | 100 | 100 | 100 |
| Reduced (%) | | | | |
| Purity (SDS-PAGE), | 100 | 100 | 100 | 100 |
| Non-reduced (%) | | | | |
| Purity (SEC-HPLC) (%) | 99.1 | 99.0 | 99.1 | 99.1 |
| Potency (Esterase | 113 (23.4 | 127 (26.1 | 125 (25.8 | 129 (26.6 |
| Activity) (%) | units/mg) | units/mg) | units/mg) | units/mg) |
| Free Thiol (mol/mol) | 1.6 | 1.5 | 1.6 | 1.6 |
| Sialic Acid Content | NT | | | NT |
| (pmol/pmol) | | | | |

*Result is an average of vials taken from beginning, middle, and end of the lyo cycle

The results indicate that the TBU cycle produces pharmaceutically acceptable cakes that are white to off-white in color and intact. Post reconstitution, samples are clear and free of particulate matter. Additionally, samples up to 12 hours post-reconstitution pass acceptance criteria (Tables 15, 16).

TABLE 16
Acceptance Criteria

| Test | Analytical Method | Acceptance Criteria |
|---|---|---|
| Appearance | Visual inspection | White to off-white cake |
| (pre-reconstitution) | | |
| Reconstitution time | | Report results (at minutes: ≦1 min |
| (reconstitute with 1.0 mL | | if time needed is less than one |
| WFI) | | minute) |
| Appearance | | Clear to opalescent, pale yellow to |
| (post-reconstitition) | | yellow solution, essentially free |
| | | from foreign particulate matter |
| pH | pH Electrode | 7.2 ± 0.4 |
| | USP <791> | |
| | Ph. Eur. 2.2.3 | |
| Osmolality | Freezing point | 300 ± 50 mOsm/kg |
| | USP <785> | |
| | Ph. Eur. 2.2.35 | |
| Purity | SDS-PAGE: Reduced | ≧90% |
| | and non-reduced with | |
| | Coomassie blue stain | |
| | SDS-PAGE: Reduced | Comparable to reference standard |
| | and non-reduced with | |
| | Silver stain | |
| | SE-HPLC | ≧90% |
| Potency | Esterase Assay | 15-29 units/mg protein |
| Identity | ELISA | Identity confirmed |
| Protein concentration | Absorbance at 280 nm | 100.0 ± 20.0 mg/mL |
| (average of three values | | |
| reported) | | |
| Sterility | USP <71> | No growth |
| | Ph. Eur. 2.6.1 | |
| Bacterial Endotoxin | Kinetic turbidimetric | ≦1.200 EU/mg |
| | USP <85> | |
| | Ph. Eur. 2.6.14 | |
| Subvisible Particulate Matter | Light Obscuration | ≧10 μm NMT 6000 part/container |
| | USP <788> | ≧25 μm NMT 600 part/container |
| | Ph. Eur. 2.9.19 | |
| Residual Moisture (Three | Karl-Fischer | ≦3.0% |
| individual values reported) | Coulometer | |

### Conclusion of Lyophilization Evaluation

For essentially equivalent formulations, it is preferable to use the formulation containing a lower concentration of salt for the lyophilization process. Therefore, the P50MTT formulation was selected as the final concentrated product formulation and was used for the lyophilization formulation evaluation and long term stability program.

To summarize the lyophilization evaluation studies, the TBU lyophilization cycle is appropriate for the lyophilization of Composition 1. The results of the low thermal analysis study indicate that the parameters of the TBU lyophilization cycle meet the minimum temperature requirements and the pre and post lyophilization results suggest that there is no change in protein quality. Cakes produced using the TBU lyophilization cycle are white to off-white in color and are intact, which is considered to be pharmaceutically acceptable.

The results of the lyophilization evaluation also suggest that the P50MTT is an appropriate formulation for the concentrated product. Upon reconstitution, samples remain clear and free of particulate matter.

### Conclusion

The formulation studies were executed to determine an appropriate formulation for the lyophilized concentrated product.

The pre-formulation studies demonstrated that increasing ionic strength results in a significant reduction in dose dependent aggregation at a protein concentration of 100 mg/ml. PS80 concentration had no significant effect and a concentration of 0.03% was selected for use in the proto-formulations.

Two proto-formulations, P50MTT and P60MTT, were selected for additional studies.

The study results indicates that Composition 1 drug substances at 100 mg/mL with these two formulations are stable at 2-8° C. and 25° C. for up to 6 days, and are neither sensitive to freeze-thaw nor shaking effects, which could support the lyophilization process. There is no significant impact on the product quality by post-lyophilization. Overall, the two formulations are comparable in terms of the product quality and stability.

However, the P50MTT formulation was selected as a formulation candidate for an additional lyophilization cycle evaluation and long term stability study, due to its lower ionic strength compared to P60MTT, which might negatively impact lyophilization process and lyophilization product.

The lyophilization evaluation studies support that the TBU cycle produces pharmaceutically acceptable cakes.

Overall, the results of the formulation studies demonstrate that P50MTT is a suitable lyophilization formulation for the concentrated product and the TBU lyophilization program would be an appropriate lyophilization process to use for concentrated product fill.

### Example 2

### Long Term Stability Testing of Composition 1

### Methods

Composition 1 (100 mg/ml in P50MTT after reconstitution with 1.1 ml of WFI) was used for the stability program study. The lyophilized product was stored at 2-8° C., 25° C. and 40° C.

### Results

At the end of 6 months, there is no significant change in SE-HPLC purity for Composition 1 when stored at 2-8° C. (Table 17). The quality attributes of samples stored at the recommended conditions meet all acceptance criteria to at least 6 months. When stored at elevated temperature conditions, such as 25° C. and 40° C., there is a 2.5% and 9.6% loss in SE-HPLC purity after 6 months, respectively (Tables 18 and 19). However, potency was within tolerances for all temperature conditions up to 6 months (Tables 17, 18 and 19).

TABLE 17
Stability Data for Composition 1 When Stored at
Recommended Conditions, 2-8° C.

| | | | Time (months) | | | | | | | |
|---|---|---|---|---|---|---|---|---|---|---|
| Attributes | | Acceptance Criteria | 0 | 1 | 3 | 6 | 9 | 12 | 18 | 24 |
| Appearance | | White to off-white cake | WC | WC | WC | WC | WC | WC | WC | WC |
| (Pre-reconstitution) | | | | | | | | | | |
| Appearance | | Report results (≦ X min) | 6 min | 7 min | 4 min | 6 min | 5 min | 5 min | 5 min | 5 min |
| (Reconstitution time) | | | | | | | | | | |
| Appearance | | Clear to opalescent, pale | CYF | CYF | CYF | CYF | CYF | CYF | CYF | CYF |
| (Post-reconstitution) | | yellow to yellow solution, | | | | | | | | |
| | | essentially free from | | | | | | | | |
| | | foreign particulate matter | | | | | | | | |
| pH | | 7.2 ± 0.4 | 7.2 | 7.2 | 7.2 | 7.1 | 7.2 | 7.1 | 7.2 | 7.2 |
| Osmolality (Freezing | | 300 ± 50 mOsm/kg | 287 | 288 | 283 | 284 | 292 | 281 | 283 | 296 |
| point) | | | | | | | | | | |
| Protein Concentration | | 100 ± 20 mg/mL | 100.2¹ | 95.8 | 96.7 | 96.5 | 97.4 | 104.5 | 102.5 | 100.8 |
| (A₂₈₀) | | | | | | | | | | |
| Purity | Reduced | ≧90.0% | 100 | 100 | 100 | 100 | 99 | 99 | 98 | 99 |
| (SDS- | Non- | ≧90.0% | 100 | 100 | 100 | 100 | 99 | 99 | 98 | 99 |
| PAGE), | reduced | | | | | | | | | |
| Coomassie | | | | | | | | | | |
| Purity | Main | ≧90% | 99.4 | 99.3 | 99.2 | 99.1 | 98.9 | 98.9 | 98.7 | 98.6 |
| (SEC- | Peak | | | | | | | | | |
| HPLC) | RRT | Report Results (X.X %) | 0.0 | 0.0 | 0.0 | 0.0 | 0.0 | 0.0 | 0.0 | 0.0 |
| | 0.60-0.78 | | | | | | | | | |
| | RRT | Report Results (X.X %) | 0.3 | 0.4 | 0.5 | 0.6 | 0.7 | 0.8 | 1.0 | 1.1 |
| | 0.87 | | | | | | | | | |
| | RRT | Report Results (X.X %) | 0.3 | 0.3 | 0.3 | 0.3 | 0.3 | 0.3 | 0.3 | 0.3 |
| | 1.09-1.28 | | | | | | | | | |
| Potency (Esterase Assay) | | 15-29 units/mg protein | 23.9 | 21.2 | 20.7 | 24.1 | 24.1 | 20.1 | 23 | 24 |
| Residual Moisture | | ≦3.0% | 0.4¹ | 0.5 | 0.7 | 0.6 | 0.9 | 0.8 | 0.6 | 0.8 |
| Purity | Main | Report Results (X.X %) | 89.5 | 89.5 | 89.2 | 89.5 | 89.6 | 89.3 | 89.5 | 89.7 |
| (HIC-HPLC) | Peak | | | | | | | | | |
| | RRT | Report Results (X.X %) | 9.1 | 9.0 | 9.1 | 9.2 | 9.1 | 9.0 | 9.1 | 9.0 |
| | 1.09 | | | | | | | | | |

| | | | | | |
|---|---|---|---|---|---|
| Free Thiol (Ellman's | Report Results (X.X | 1.5 | 1.8 | 1.5 | 1.7 |
| Assay) | mol/mol) | | | | |
| Sialic Acid Content | Report Results (X.X | 8.8 | | 9.7 | x |
| | pmol/pmol) | | | | |
| Deamidation | Report Results (X.X | NT | | 0.0175 | 0.0193 |
| | pmol/pmol) | | | | |

WC = White Cake,
CYF = Clear, Yellow solution, essentially free from foreign particulate matter
¹Result is an average of 3 vials (1 each from beginning, middle, and end)

TABLE 18
Stability Data for Composition 1, 25° C.

| | | Time (months) | | | | | |
|---|---|---|---|---|---|---|---|
| Attributes | | 0 | 1 | 3 | 6 | 9 | 12 |
| Appearance (Pre-reconstitution) | | WC | WC | WC | WC | WC | WC |
| Appearance (Reconstitution time) | | 6 min | 8 min | 5 min | 6 min | 5 min | 4 min |
| Appearance (Post-reconstitution) | | CYF | CYF | CYF | CYF | CYF | CYF |
| pH | | 7.2 | 7.2 | 7.2 | 7.1 | 7.2 | 7.2 |
| Osmolality (Freezing point) (mOsm/kg) | | 287 | 287 | 296 | 288 | 285 | 294 |
| Protein Concentration (A₂₈₀) (mg/mL) | | 100.2¹ | 96.8 | 95.2 | 96.4 | 98.5 | 102.7 |
| Purity | Reduced (%) | 100 | 99 | 99 | 98 | 97 | 98 |
| (SDS-PAGE), | Non-reduced (%) | 100 | 100 | 99 | 98 | 97 | 96 |
| Coomassie | | | | | | | |
| Purity | Main Peak (%) | 99.4 | 98.6 | 97.8 | 97.1 | 96.4 | 95.9 |
| (SEC-HPLC) | RRT 0.60-0.78 (%) | 0.0 | 0.0 | 0.0 | 0.0 | 0.1 | 0.0 |
| | RRT 0.87 (%) | 0.3 | 1.1 | 1.8 | 2.5 | 3.2 | 3.8 |
| | RRT 1.09-1.28 (%) | 0.3 | 0.3 | 0.3 | 0.3 | 0.3 | 0.4 |
| Potency (Esterase Assay) | | 23.9 | 20.7 | 24.0 | 23.1 | 24.2 | 18.0 |
| (units/mg protein) | | | | | | | |
| Purity | Main Peak (%) | 89.5 | 89.6 | 88.9 | 89.4 | 89.8 | 89.4 |
| (HIC-HPLC) | RRT 1.09 (%) | 9.1 | 8.9 | 9.2 | 9.2 | 8.6 | 8.6 |

| | | | |
|---|---|---|---|
| Free Thiol (Ellman's Assay) (mol/mol) | 1.5 | 1.8 | 1.5 |
| Sialic Acid Content (pmol/pmol) | 8.8 | | 10.1 |
| Deamidation (pmol/pmol) | NT | | 0.0169 |

WC = White Cake,
CYF = Clear, Yellow solution, essentially free from foreign particulate matter
¹Result is an average of 3 vials (1 each from beginning, middle, and end)

TABLE 19
Stability Data for Composition 1, 40° C.

| | | Time (months) | | | |
|---|---|---|---|---|---|
| Attributes | | 0 | 1 | 3 | 6 |
| Appearance (Pre-reconstitution) | | WC | WC | WC | WC |
| Appearance (Reconstitution time) | | 6 min | 8 min | 5 min | 6 min |
| Appearance (Post- reconstitution) | | CYF | CYF | CYF | CYF |
| pH | | 7.2 | 7.2 | 7.2 | 7.1 |
| Osmolality (Freezing point) (mOsm/kg) | | 287 | 283 | 283 | 288 |
| Protein Concentration (A₂₈₀) (mg/mL) | | 100.2¹ | 95.8 | 94.4 | 96.4 |
| Purity | Reduced (%) | 100 | 97 | 93 | 93 |
| (SDS-PAGE), | Non-reduced (%) | 100 | 97 | 94 | 93 |
| Coomassie | | | | | |
| Purity | Main Peak (%) | 99.4 | 96.4 | 93.8 | 91.1 |
| (SEC-HPLC) | RRT 0.60-0.78 (%) | 0.0 | 0.0 | 0.3 | 0.6 |
| | RRT 0.87 (%) | 0.3 | 1.1 | 5.6 | 8.0 |
| | RRT 1.09-1.28 (%) | 0.3 | 0.3 | 0.4 | 0.3 |
| Potency (Esterase Assay) (units/mg | | 23.9 | 21.1 | 20.7 | 23.7 |
| protein) | | | | | |
| Purity | Main Peak (%) | 89.5 | 89.7 | 89.0 | 88.4 |
| (HIC-HPLC) | RRT 1.09 (%) | 9.1 | 8.8 | 8.6 | 9.3 |

| | | |
|---|---|---|
| Free Thiol (Ellman's Assay) | 1.5 | 1.7 |
| (mol/mol) | | |

WC = White Cake,
CYF = Clear, Yellow solution, essentially free from foreign particulate matter
¹Result is an average of 3 vials (1 each from beginning, middle, and end)

Samples stored under recommended conditions are stable under the recommended conditions for 18 months.

Samples stored under recommended conditions are stable for 24 months.

Samples stored under recommended conditions are stable for 36 months.

### Example 3

### Long Term Stability Testing of Composition 2

### Methods

Composition 2, known as Neugranin™, is a protein derived from the direct genetic fusion of the genes for Granulocyte Colony Stimulating Factor (GCSF) and human serum albumin. The TBU lypholization cycle applied to Composition 2 (15 mg/ml) is summarized in Table 20. The lyophilized product was stored at 2-8° C., 25° C. and 40° C.

### Results

At the end of 6 months, there is no significant change in SEC-HPLC purity for Composition 2 when stored at 2-8° C. (Table 21). The quality attributes of samples stored at the recommended conditions meet all acceptance criteria to at least 6 months. When stored at elevated temperature conditions, such as 25° C. and 40° C., there is a 0.4% gain and a 0.1% loss in SEC-HPLC purity after 6 months, respectively (Tables 22-23). However, potency was within tolerances for all temperature conditions up to 6 months (Tables 21, 22 and 23).

TABLE 20
TBU Lyophilization Cycle

| Step | Parameters |
|---|---|
| a | Pre-cool shelves to 5° C. |
| b | Load product and hold for at least 2 hours at 5° C. |
| c | Cool shelf temperature at 0.3° C./min to −45° C. |
| d | Hold for 3 hours at −45° C. |
| e | Warm shelf temperature at 0.8° C./min to −18° C. |
| f | Hold for 5 hours at −18° C. (annealing/thermal treatment). |
| g | Cool shelf temperature −45° C. at 0.3° C./min. |
| h | Hold at −45° C. for 2 hours. |
| i | Engage vacuum and adjust to 100 mT as controlled by capacitance |
| | manometer. |
| j | Hold shelf temperature at −45° C. until vacuum set point is |
| | achieved. |
| k | Hold at −45° C. for 1 hour. |
| l | Warm shelf temperature to −10° C. at 0.8° C./min. |
| m | Hold shelf temperature at −10° C. for 36 hours (primary drying). |
| n | Ensure that all functioning product thermocouples have been at or |
| | above −10° C. for at least 3 hours before proceeding to the next |
| | step. If not, continue to hold the shelf temperature at −10° C. until |
| | all functioning product thermocouples have been at or |
| | above −10° C. for at least 3 hours. |
| o | Warm shelf temperature to 25° C. at 0.8° C./min. |
| p | Hold at 25° C. for 15 hours (secondary drying). |
| q | At end of secondary drying, slowly restore chamber to |
| | approximately 600 T with sterile filtered nitrogen, NF/EP and |
| | seat stoppers. |

TABLE 21
Stability Data for Composition 2 When Stored at
Recommended Conditions, 2-8° C.

| | | Acceptance | Time (Months) | | | | | | | | |
|---|---|---|---|---|---|---|---|---|---|---|---|
| Attributes | | Criteria | 0 | 1 | 3 | 6 | 8 | 12 | 18 | 24 | 30 |
| Appearance | | White to off- | WC | WC | WC | WC | WC | WC | WC | WC | WC |
| (Pre-reconstitution) | | white cake | | | | | | | | | |
| Appearance | | Report results | 20 | 21 | 24 | 26 | 26 | 29 | 26 | ≦1 min | ≦1 min |
| (Reconstitution | | (sec) | | | | | | | | | |
| time) | | | | | | | | | | | |
| Appearance | | Clear to | CPF | CPF | CPF | CPF | CPF | CPF | CPF | CPF | CPF |
| (Post- | | opalescent, pale | | | | | | | | | |
| reconstitution) | | yellow to yellow, | | | | | | | | | |
| | | essentially free | | | | | | | | | |
| | | from foreign | | | | | | | | | |
| | | particulate matter | | | | | | | | | |
| pH | | 7.2 ± 0.4 | 7.3 | 7.2 | 7.3 | 7.3 | 7.3 | 7.3 | 7.3 | 7.2 | 7.2 |
| Osmolality | | Report results | 329 | 360 | 299 | 296 | 313 | 318 | 307 | 312 | 303 |
| (Freezing point) | | (mOsm/kg) | | | | | | | | | |
| Protein | | 15.0 ± 3.0 mg/mL | 16.1 | 16.3 | 15.4 | 17.1 | 15.8 | 15.7 | 15.6 | 15.4 | 14.7 |
| Concentration (A₂₈₀) | | | | | | | | | | | |
| (mg/ml) | | | | | | | | | | | |
| Purity | Reduced | ≧90.0% | 100 | 100 | 100 | 100 | 100 | 100 | 100 | 100 | 100 |
| (SDS- | (%) | | | | | | | | | | |
| PAGE) | Non- | ≧90.0% | 100 | 100 | 100 | 100 | 100 | 100 | 100 | 100 | 100 |
| | reduced | | | | | | | | | | |
| | (%) | | | | | | | | | | |
| Purity (SEC-HPLC) | | ≧90.0% | 97.6 | 96.3 | 97.9 | 98.2 | 97.7 | 96.2 | 97.6 | 96.5 | 96.8 |
| Purity | MP (%) | Report Results | 86.5 | 87.7 | 87.5 | 87.5 | 86.6 | 85.4 | 84.8 | 84.9 | 84.0 |
| (RP- | | (%) | | | | | | | | | |
| HPLC) | RRT | Report Results | 10.2 | 10.1 | 10.1 | 10.5 | 10.3 | 10.6 | 10.8 | 10.2 | 10.2 |
| | 0.98 (%) | (%) | | | | | | | | | |
| | MP+ | Report Results | 96.7 | 97.8 | 97.6 | 98.0 | 96.9 | 95.9 | 95.5 | 95.0 | 94.3 |
| | RRT .98 | (%) | | | | | | | | | |
| | (%) | | | | | | | | | | |

| | | | | | | | | | | |
|---|---|---|---|---|---|---|---|---|---|---|
| Purity (IEC-HPLC) | FIO (%) | NT | NT | NT | NT | NT | 78.5 | 72.6 | 75.8 | 73.0 |
| Potency (bioassay) | Report Results | 134 | 114 | 93 | 99.1 | 114 | 110 | 84.3 | 113 | 123 |
| (relative potency %) | (%) | | | | | | | | | |
| Residual Moisture | ≦3.0% | 0.4 | 0.3 | 0.3 | 1.1 | 0.3 | 0.3 | 0.2 | 0.5 | 0.3 |

WC = White Cake,
CPF = Clear, pale yellow, essentially free from foreign particulate matter;
FIO—For information only;
NT—Not Test

TABLE 22
Stability Data for Composition 2, 25° C.

| | | Time (months) | | | | | |
|---|---|---|---|---|---|---|---|
| Attributes | | 0 | 1 | 3 | 6 | 8 | 12 |
| Appearance | | WC | WC | WC | WC | WC | WC |
| (Visual inspection- pre | | | | | | | |
| reconstitution; cake) | | | | | | | |
| Appearance | | 20 | 21 | 24 | 23 | 18 | 30 |
| (Visual inspection- | | | | | | | |
| reconstitution time | | | | | | | |
| seconds) | | | | | | | |
| Appearance | | CPF | CPF | CPF | CPF | CPF | CPF |
| (Post-reconstitution) | | | | | | | |
| pH | | 7.3 | 7.2 | 7.3 | 7.3 | 7.3 | 7.3 |
| Osmolality (Freezing | | 329 | 334 | 321 | 302 | 309 | 310 |
| point) | | | | | | | |
| Protein Concentration | | 16.1 | 16.4 | 16.3 | 16.1 | 15.6 | 15.2 |
| (A₂₈₀) (mg/ml) | | | | | | | |
| Purity | Reduced | 100 | 100 | 100 | 100 | 100 | 100 |
| (SDS- | (%) | | | | | | |
| PAGE) | Non- | 100 | 100 | 100 | 100 | 100 | 100 |
| | reduced | | | | | | |
| | (%) | | | | | | |
| Purity (SEC-HPLC) | | 97.6 | 96.4 | 97.8 | 98.0 | 97.5 | 95.7 |
| (%) | | | | | | | |
| Purity | MP (%) | 86.5 | 87.5 | 86.9 | 87.4 | 86.3 | 84.4 |
| (RP- | RRT | 10.2 | 10.1 | 10.2 | 10.5 | 10.1 | 10.8 |
| HPLC) | 0.98 (%) | | | | | | |
| | MP+ | 96.7 | 97.7 | 97.1 | 97.9 | 96.4 | 95.2 |
| | RRT .98 | | | | | | |
| | (%) | | | | | | |

| | | | | | | |
|---|---|---|---|---|---|---|
| Purity (IEC-HPLC) | NT | NT | NT | NT | NT | 77.1 |
| (%) | | | | | | |
| Potency (bioassay) (%) | 134 | 119 | 115 | 122 | 105 | 115 |
| Residual Moisture (%) | 0.4 | 0.5 | 0.5 | 0.5 | 0.5 | 0.7 |

WC = White Cake,
CPF = Clear, pale yellow, essentially free from foreign particulate matter;
NT—Not tested

TABLE 23
Stability Data for Composition 2, 40° C.

| | | Time (months) | | | | | |
|---|---|---|---|---|---|---|---|
| Attributes | | 0 | 1 | 3 | 6 | 8 | 12 |
| Appearance | | WC | WC | WC | WC | WC | WC |
| (Pre-reconstitution) | | | | | | | |
| Appearance | | 20 | 17 | 25 | 24 | 26 | 30 |
| (Reconstitution time) | | | | | | | |
| Appearance | | CPF | CPF | CPF | CPF | CPF | CPF |
| (Post-reconstitution) | | | | | | | |
| pH | | 7.3 | 7.3 | 7.3 | 7.3 | 7.3 | 7.3 |
| Osmolality (Freezing | | 329 | 335 | 315 | 298 | 309 | 309 |
| point) | | | | | | | |
| Protein Concentration | | 16.1 | 16.7 | 16.1 | 15.6 | 15.8 | 15.3 |
| (A₂₈₀) | | | | | | | |
| Purity | Reduced | 100 | 100 | 100 | 100 | 100 | 99 |
| (SDS- | Non- | 100 | 100 | 100 | 100 | 100 | 99 |
| PAGE) | reduced | | | | | | |
| Purity (SEC-HPLC) | | 97.6 | 96.1 | 97.4 | 97.5 | 96.8 | 94.4 |
| Purity | MP | 86.5 | 87.3 | 87.0 | 87.0 | 85.6 | 83.0 |
| (RP- | RRT | 10.2 | 10.3 | 10.2 | 10.8 | 10.5 | 11.1 |
| HPLC) | 0.98 | | | | | | |
| | MP+ | 96.7 | 97.5 | 97.2 | 97.8 | 96.1 | 94.1 |
| | RRT .98 | | | | | | |

| | | | | | | |
|---|---|---|---|---|---|---|
| Purity (IEC-HPLC) | NT | NT | NT | NT | NT | 69.4 |
| Potency (bioassay) | 134 | 122 | 101 | 107 | 83 | 108 |
| Residual Moisture | 0.4 | 0.7 | 0.6 | 0.7 | 1.6 | 1.3 |

WC = White Cake,
CPF = Clear, pale yellow, essentially free from foreign particulate matter;
NT—Not tested

Samples stored under recommended conditions are stable under the recommended conditions for 18 months.

Samples stored under recommended conditions are stable for 24 months.

Samples stored under recommended conditions are stable for 36 months.

### Example 4

### Long Term Stability Testing of Composition 3

### Methods

Composition 3, known as Albuferon™-Beta, is a product derived from the direct genetic fusion of the genes for human interferon-beta (IFN-beta) and human serum albumin. The TBU lypholization cycle applied to Composition 3 (2.0 mg/ml) is summarized in Table 24. The lyophilized product was stored at 2-8° C., 25° C. and 40° C.

### Results

At the end of 6 months, there is no significant change in SEC-HPLC purity for Composition 3 when stored at 2-8° C. (Table 25). The quality attributes of samples stored at the recommended conditions meet all acceptance criteria to at least 6 months. When stored at elevated temperature conditions, such as 25° C. and 40° C., there is a 1.0% and a 3.1% loss in SEC-HPLC purity after 6 months, respectively (Tables 26-27). However, potency was within tolerances for all temperature conditions up to 6 months (Tables 25, 26 and 27).

TABLE 24
TBU Lyophilization Cycle

| Step | Parameters |
|---|---|
| a | Pre-cool shelves to 5° C. |
| b | Load product and hold for at least 2 hours at 5° C. |
| c | Cool shelf temperature at 0.3° C./min to −45° C. |
| d | Hold for 3 hours at −45° C. |
| e | Warm shelf temperature at 0.8° C./min to −18° C. |
| f | Hold for 5 hours at −18° C. (annealing/thermal treatment). |
| g | Cool shelf temperature to −45° C. at 0.3° C./min. |
| h | Hold at −45° C. for 2 hours. |
| i | Engage vacuum and adjust to 100 mTorr as controlled by |
| | capacitance manometer. |
| j | Hold shelf temperature at −45° C. until vacuum set point is |
| | achieved. |
| k | Hold at −45° C. for 1 hour. |
| l | Warm shelf temperature to −10° C. at 0.8° C./min. |
| m | Hold shelf temperature at −10° C. for 36 hours (primary drying). |
| n | Ensure that all functioning product thermocouples have been at or |
| | above −10° C. for at least 3 hours before proceeding to the next |
| | step. If not, continue to hold the shelf temperature at −10° C. until |
| | all functioning product thermocouples have been at or |
| | above −10° C. for at least 3 hours. |
| o | Warm shelf temperature to 25° C. at 0.8° C./min. |
| p | Hold at 25° C. for 2.0 hours (secondary drying). |
| q | At end of secondary drying, slowly restore chamber to |
| | approximately 600 Torr with sterile filtered nitrogen, NF/EP and |
| | seat stoppers. |

TABLE 25
Stability Data for Composition 3 When Stored at
Recommended Conditions, 2-8° C.

| | | | Time (months) | | | | | |
|---|---|---|---|---|---|---|---|---|
| Attributes | | Acceptance Criteria | 0 | 1 | 3 | 6 | 9 | 12 |
| Appearance | | White to off-white cake | WC | WC | WC | WC | WC | WC |
| (Pre-reconstitution) | | | | | | | | |
| Appearance | | Report results (≦X min) | 1 min | 1 min | 1 min | 1 min | 1 min | 1 min |
| (Reconstitution time) | | | | | | | | |
| Appearance | | Clear, pale yellow to | CPF | CPF | CPF | CPF | CPF | CPF |
| (Post- reconstitution) | | yellow solution essentially | | | | | | |
| | | free from foreign | | | | | | |
| | | particulate matter | | | | | | |
| pH | | 7.2 ± 0.4 | 7.2 | 7.1 | 7.2 | 7.2 | 7.2 | 7.2 |
| Osmolality (Freezing | | 300 ± 50 mOsm/kg | 299 | 299 | 306 | 298 | 309 | 307 |
| point) | | | | | | | | |
| Protein Concentration | | 2.0 ± 0.4 mg/mL | 2.0¹ | 2.0 | 2.0 | 2.0 | 2.0 | 2.1 |
| (A₂₈₀) (mg/ml) | | | | | | | | |
| Purity | Reduced | ≧95.0% | 98 | 98 | 97 | 99 | 98 | 100 |
| (SDS- | (%) | | | | | | | |
| PAGE) | Non- | ≧95.0% | 100 | 100 | 99 | 99 | 97 | 100 |
| | reduced | | | | | | | |
| | (%) | | | | | | | |

| | | | | | | | |
|---|---|---|---|---|---|---|---|
| Purity (SE-HPLC) (%) | ≧90.0% | 98.4 | 98.5 | 98.2 | 98.1 | 98.3 | 97.4 |
| Purity | Report Results (X.X %) | 92.6 | 90.8 | 92.2 | 92.4 | 91.5 | 92.8 |
| (RP-HPLC) (%) | | | | | | | |
| Potency (bioassay) (%) | Report Results (X %) | 90 | 71.8 | 93 | 84 | 89.2 | 85.5 |
| Residual Moisture (%) | ≦3.0% | 1.1 | 1.4 | 1.0 | 1.8 | 1.3 | 2.0 |
| Deamidation | Report Results | NT | 0.30 | 0.34 | 0.36 | 0.256 | |
| (pmol/pmol) | (X.X pmol/pmol) | | | | | | |
| Bioburden (Membrane | ≦10 CFU/10 mL | 0 CFU/ | | | | | |
| Filtration) | | 10 mL | | | | | |

¹Result is an average of vials taken from beginning, middle, and end of the lyo cycle.
WC = White Cake,
CPF = Clear, pale yellow solution, essentially free from foreign particulate matter;
FIO—For information only;
NT—Not tested

TABLE 26
Stability Data for Composition 3, 25° C.

| | | Time (months) | | | | | |
|---|---|---|---|---|---|---|---|
| Attributes | | 0 | 1 | 3 | 6 | 9 | 12 |
| Appearance | | WC | WC | WC | WC | WC | WC |
| (Pre-reconstitution) | | | | | | | |
| Appearance | | 1 min | 1 min | 1 min | 1 min | 1 min | 1 min |
| (Reconstitution time) | | | | | | | |
| Appearance | | CPF | CPF | CPF | CPF | CPF | CPF |
| (Post-reconstitution) | | | | | | | |
| pH | | 7.2 | 7.1 | 7.2 | 7.2 | 7.2 | 7.1 |
| Osmolality (Freezing | | 299 | 303 | 307 | 296 | 312 | 315 |
| point) | | | | | | | |
| Protein Concentration | | 2.0¹ | 2.1 | 2.1 | 2.0 | 2.1 | 2.1 |
| (A₂₈₀) (mg/ml) | | | | | | | |
| Purity | Reduced | 98 | 98 | 98 | 99 | 98 | 100 |
| (SDS- | (%) | | | | | | |
| PAGE) | Non- | 100 | 99 | 98 | 99 | 97 | 100 |
| | reduced | | | | | | |
| | (%) | | | | | | |

| | | | | | | |
|---|---|---|---|---|---|---|
| Purity (SE-HPLC) (%) | 98.4 | 97.5 | 96.8 | 97.4 | 96.9 | 95.9 |
| Purity | 92.6 | 90.7 | 91.0 | 88.9 | 91.4 | 89.9 |
| (RP-HPLC) (%) | | | | | | |
| Potency (bioassay) (%) | 90 | 81 | 120 | 86 | 74.8 | 101 |
| Residual Moisture (%) | 1.1 | 1.7 | 1.0 | 2.1 | 2.1 | 1.3 |
| Deamidation | NT | 0.29 | 0.31 | 0.37 | 0.246 | |
| (pmol/pmol) | | | | | | |

¹Result is an average of vials taken from beginning, middle, and end of the lyo cycle.
WC = White Cake,
CPF = Clear, pale yellow, essentially free from foreign particulate matter;
NT—Not tested

TABLE 27
Stability Data for Composition 3, 40° C.

| | | Time (months) | | | |
|---|---|---|---|---|---|
| Attributes | | 0 | 1 | 3 | 6 |
| Appearance (Pre-reconstitution) | | WC | WC | WC | WC |
| Appearance (Reconstitution time) | | 1 min | 1 min | 1 min | 1 min |
| Appearance (Post- reconstitution) | | CPF | CPF | CPF | CPF |
| pH | | 7.2 | 7.1 | 7.2 | 7.2 |
| Osmolality (Freezing point) | | 299 | 303 | 316 | 301 |
| Protein Concentration (A₂₈₀) | | 2.0 ¹ | 2.1 | 2.1 | 2.0 |
| (mg/ml) | | | | | |
| Purity | Reduced (%) | 98 | 98 | 98 | 98 |
| (SDS-PAGE) | Non-reduced (%) | 100 | 100 | 98 | 99 |

| | | | | |
|---|---|---|---|---|
| Purity (SE-HPLC) (%) | 98.4 | 97.2 | 95.8 | 95.3 |
| Purity (RP-HPLC) (%) | 92.6 | 89.7 | 87.4 | 85.0 |
| Potency (bioassay) (%) | 90 | 79 | 83 | 82 |
| Residual Moisture (%) | 1.1 | 1.2 | 1.5 | 1.9 |
| Deamidation (pmol/pmol) | NT | 0.31 | 0.32 | 0.39 |

¹ Result is an average of vials taken from beginning, middle, and end of the lyo cycle.
WC = White Cake,
CPF = Clear, pale yellow, essentially free from foreign particulate matter;
NT—Not tested

Samples stored under recommended conditions are stable under the recommended conditions for 18 months.

Samples stored under recommended conditions are stable for 24 months.

Samples stored under recommended conditions are stable for 36 months.

### Example 5

### Long Term Stability Testing of Composition 4

### Methods

Composition 4, known as Albutropin™, is a contiguous protein comprised of human serum albumin (HSA) and recombinant growth hormone (rHGH) with the mature form of HSA genetically fused at its C-terminus to the N-terminus of the mature form of rHGH. The TBU lypholization cycle applied to Composition 4 (25.0 mg/ml) is summarized in Table 28. The lyophilized product was stored at 2-8° C., 25° C. and 40° C.

### Results

At the end of 6 months, there is no significant change in SEC-HPLC purity for Composition 4 when stored at 2-8° C. (Table 29). The quality attributes of samples stored at the recommended conditions meet all acceptance criteria to at least 6 months. When stored at elevated temperature conditions, such as 25° C. and 40° C., there is a 0.8% and a 2.6% loss in SEC-HPLC purity after 6 months, respectively (Tables 30-31). However, potency was within tolerances for all temperature conditions up to 6 months (Tables 29, 30 and 31).

TABLE 28
TBU Lyophilization Cycle

| Step | Parameters |
|---|---|
| a | Pre-cool shelves to 5° C. |
| b | Load product and hold for at least 2 hours at 5° C. |
| c | Cool shelf temperature at 0.3° C./min to −45° C. and hold for 5 |
| | hours at −45° C. |
| d | Engage vacuum and adjust to 100 mT as controlled by capacitance |
| | manometer |
| e | Hold shelf temperature at −45° C. until vacuum set point is |
| | achieved. |
| f | Hold at −45° C. for 1 hour |
| g | Warm shelf temperature to −10° C. at 0.8° C./min and hold shelf |
| | temperature at −10° C. for 36 hours (primary drying) |
| h | Ensure that all functioning product thermocouples have been at or |
| | above −10° C. for at least 3 hours before proceeding to the next |
| | step. If not, continue to hold the shelf temperature at −10° C. |
| | until all functioning product thermocouples have been at or |
| | above −10° C. for at least 3 hours |
| i | Warm shelf temperature to 25° C. at 0.8° C./min and hold at |
| | 25° C. for 15 hours at 100 mT (secondary drying) |
| j | Ramp to 5° C. for 40 min and hold for secondary drying for 5 |
| | hours at 100 mTorr |
| k | At end of secondary drying, slowly restore chamber to |
| | approximately 810 mBar with sterile filtered nitrogen, NF/EP and |
| | seat stoppers. |

TABLE 29
Stability Data for Composition 4 When Stored at
Recommended Conditions, 2-8° C.

| | | | Time (months) | | | | | | |
|---|---|---|---|---|---|---|---|---|---|
| Attributes | | Acceptance Criteria | 0 | 1 | 3 | 6 | 9 | 12 | 17 |
| Appearance | | White to off-white cake | WC | WC | WC | WC | WC | WC | WC |
| (Pre-reconstitution) | | | | | | | | | |
| Appearance | | Report results (≦X min) | 1 min | 1 min | 1 min | 1 min | 1 min | 1 min | 1 min |
| (Reconstitution time) | | | | | | | | | |
| Appearance | | Report results | CPF | CPF | CPF | CPF | CPF | CPF | CPF |
| (Post-reconstitution) | | | | | | | | | |
| pH | | 7.2 ± 0.4 | 7.1 | 7.2 | 7.2 | 7.2 | 7.1 | 7.2 | |
| Osmolality (Freezing | | 300 ± 50 mOsm/kg | 311 | 307 | 305 | 313 | 303 | 309 | |
| point) | | | | | | | | | |
| Protein Concentration | | 25 ± 5 mg/mL | 23.6¹ | 23.4 | 23.7 | 23.3 | 23.5 | 22.9 | |
| (A₂₈₀) (mg/ml) | | | | | | | | | |
| Purity | Reduced | ≧90.0% | 100 | 100 | 100 | 100 | 100 | 100 | |
| (SDS- | Non-reduced | ≧90.0% | 100 | 100 | 100 | 100 | 100 | 100 | |
| PAGE), | | | | | | | | | |
| Coomassie | | | | | | | | | |
| Purity | Main Peak | ≧92.5% | 99.1 | 98.8 | 99.0 | 98.9 | 99.0 | 98.8 | |
| (SEC- | RRT ≦0.78 | Report Results (X.X %) | 0.2 | 0.1 | 0.2 | 0.2 | 0.1 | 0.2 | |
| HPLC) | RRT 0.86 | Report Results (X.X %) | 0.4 | 0.4 | 0.5 | 0.6 | 0.5 | 0.6 | |
| | RRT 1.09 | Report Results (X.X %) | 0.3 | 0.7 | 0.3 | 0.4 | 0.4 | 0.4 | |
| Purity | Main Peak | ≧87.5% | 95.3 | 95.0 | 95.1 | 93.5 | 94.2 | 93.4 | |
| (RP- | RRT 0.64 | Report Results (X.X %) | 0.3 | 0.3 | 0.3 | 0.3 | 0.3 | 0.2 | |
| HPLC) | RRT 0.88-0.91 | Report Results (X.X %) | 0.3 | 0.3 | 0.3 | 0.4 | 0.3 | 0.3 | |
| | RRT 0.96-0.99 | Report Results (X.X %) | 0.2 | 0.3 | 0.3 | 0.3 | 0.3 | 0.2 | |
| | RRT 1.03-1.06 | Report Results (X.X %) | 2.2 | 2.2 | 2.2 | 2.8 | 3.0 | 2.9 | |
| | RRT 1.09-1.10 | Report Results (X.X %) | 0.4 | 0.4 | 0.4 | 0.5 | 0.4 | 0.4 | |
| | RRT 1.13 | Report Results (X.X %) | 1.3 | 1.3 | 1.3 | 2.2 | 1.6 | 2.6 | |
| | RRT 1.15 | Report Results (X.X %) | 0.0 | 0.0 | 0.0 | 0.0 | 0.0 | 0.0 | |

| | | | | | | | | |
|---|---|---|---|---|---|---|---|---|
| Potency (bioassay) | 60-140% | 111 | 105 | 105 | 113 | 107 | 127 | 119 |
| Residual Moisture | ≦3.0% | 0.2¹ | 0.4 | 0.5 | 0.5 | 0.7 | 0.7 | |
| Particulate Matter | Particles ≧10 μm: ≦6000 | 380 | | | | | 53 | |
| | particles/container | | | | | | | |
| | Particles ≧25 μm: ≦6000 | 11 | | | | | 6 | |
| | particles/container | | | | | | | |
| | Particles ≧2 μm: | | | | | | | |
| | Report Results | | | | | | | |

For Information Only

| | | | | | | | |
|---|---|---|---|---|---|---|---|
| Purity (IEC-HPLC) | FIO: (XX %) | NT | NT | NT | NT | NT | NT |
| Deamidation | Report Results | NT | NT | NT | NT | NT | 0.0078 |
| (pmol/pmol) | (X.X pmol/pmol) | | | | | | |

| | | | Time (months) | | | | |
|---|---|---|---|---|---|---|---|
| Attributes | | Acceptance Criteria | 18 | 21 | 24 | 30 | 36 |
| Appearance | | White to off-white cake | WC | WC | WC | WC | X |
| (Pre-reconstitution) | | | | | | | |
| Appearance | | Report results (≦X min) | 1 min | 1 min | 1 min | 1 min | X |
| (Reconstitution time) | | | | | | | |
| Appearance | | Report results | CPF | CPF | CPF | CPF | X |
| (Post-reconstitution) | | | | | | | |
| pH | | 7.2 ± 0.4 | 7.2 | | 7.2 | 7.2 | X |
| Osmolality (Freezing | | 300 ± 50 mOsm/kg | 301 | | 306 | 314 | X |
| point) | | | | | | | |
| Protein Concentration | | 25 ± 5 mg/mL | 24.6 | | 24.3 | 24.8 | X |
| (A₂₈₀) (mg/ml) | | | | | | | |
| Purity | Reduced | ≧90.0% | 100 | | 100 | 99 | X |
| (SDS- | Non-reduced | ≧90.0% | 100 | | 99 | 100 | X |
| PAGE), | | | | | | | |
| Coomassie | | | | | | | |
| Purity | Main Peak | ≧92.5% | 98.9 | | 98.9 | 98.8 | X |
| (SEC- | RRT ≦0.78 | Report Results (X.X %) | 0.1 | | 0.1 | 0.1 | X |
| HPLC) | RRT 0.86 | Report Results (X.X %) | 0.6 | | 0.6 | 0.7 | X |
| | RRT 1.09 | Report Results (X.X %) | 0.4 | | 0.4 | 0.4 | X |
| Purity | Main Peak | ≧87.5% | 94.3 | | 93.0 | 92.7 | X |
| (RP- | RRT 0.64 | Report Results (X.X %) | 0.3 | | 0.3 | 0.3 | X |
| HPLC) | RRT 0.88-0.91 | Report Results (X.X %) | 0.4 | | 0.3 | 1.0 | X |
| | RRT 0.96-0.99 | Report Results (X.X %) | 0.2 | | 0.4 | 0.4 | X |
| | RRT 1.03-1.06 | Report Results (X.X %) | 2.4 | | 2.5 | 2.9 | X |
| | RRT 1.09-1.10 | Report Results (X.X %) | 0.4 | | 0.5 | 0.4 | X |
| | RRT 1.13 | Report Results (X.X %) | 2.0 | | 3.0 | 2.2 | X |
| | RRT 1.15 | Report Results (X.X %) | 0.0 | | 0.0 | 0.0 | X |

| Potency (bioassay) | 60-140% | 114 | 114 | 108 | 104 | X |
|---|---|---|---|---|---|---|
| Residual Moisture | ≦3.0% | 0.8 | | 0.7 | 0.9 | X |
| Particulate Matter | Particles ≧10 μm: ≦6000 | | | 81 | | X |
| | particles/container | | | | | |
| | Particles ≧25 μm: ≦6000 | | | 2 | | X |
| | particles/container | | | | | |
| | Particles ≧2 μm: | | | 190 | | X |
| | Report Results | | | | | |
| For Information Only | | | | | | |
| Purity (IEC-HPLC) | FIO: (XX %) | NT | | X | NT | X |
| Deamidation | Report Results | 0.0080 | | 0.0063 | 0.0082 | X |
| (pmol/pmol) | (X.X pmol/pmol) | | | | | |

WC = White Cake,
CPF = Clear, Pale yellow solution, essentially free from foreign particulate matter,
NT = Not Tested,
X = Pending time point
¹Result is an average of 3 vials (1 each from beginning, middle, and end)

TABLE 30
Stability Data for Composition 4, 25° C.

| | | Time (months) | | | | | |
|---|---|---|---|---|---|---|---|
| Attributes | | 0 | 1 | 3 | 6 | 9 | 12 |
| Appearance | | WC | WC | WC | WC | WC | WC |
| (Pre-reconstitution) | | | | | | | |
| Appearance | | 1 min | 1 min | 1 min | 1 min | 1 min | 1 min |
| (Reconstitution time) | | | | | | | |
| Appearance | | CPF | CPF | CPF | CPF | CPF | CPF |
| (Post-reconstitution) | | | | | | | |
| pH | | 7.1 | 7.2 | 7.2 | 7.2 | 7.1 | 7.2 |
| Osmolality (Freezing point) | | 311 | 310 | 301 | 299 | 299 | 301 |
| Protein Concentration (A₂₈₀) | | 23.6¹ | 23.8 | 23.7 | 22.5 | 23.4 | 22.7² |
| Purity | Reduced | 100 | 100 | 100 | 100 | 100 | 99 |
| (SDS- | Non-reduced | 100 | 100 | 100 | 99 | 100 | 99 |
| PAGE), | | | | | | | |
| Coomassie | | | | | | | |
| Purity (SEC- | Main Peak | 99.1 | 98.5 | 98.6 | 98.3 | 98.2 | 98.0 |
| HPLC) | RRT ≦.78 | 0.2 | 0.2 | 0.2 | 0.2 | 0.2 | 0.2 |
| | RRT .86 | 0.4 | 0.7 | 0.8 | 1.1 | 1.2 | 1.3 |
| | RRT 1.09 | 0.3 | 0.7 | 0.4 | 0.4 | 0.4 | 0.4 |
| Purity | Main Peak | 95.3 | 94.6 | 94.4 | 92.9 | 93.0 | 92.2 |
| (RP-HPLC) | RRT .64 | 0.3 | 0.3 | 0.3 | 0.4 | 0.4 | 0.2 |
| | RRT 0.88-0.91 | 0.3 | 0.3 | 0.3 | 0.3 | 0.3 | 0.3 |
| | RRT 0.96-0.99 | 0.2 | 0.3 | 0.3 | 0.3 | 0.3 | 0.5 |
| | RRT 1.03-1.06 | 2.2 | 2.5 | 2.6 | 3.2 | 3.5 | 3.9 |
| | RRT 1.09-1.10 | 0.4 | 0.5 | 0.5 | 0.4 | 0.5 | 0.5 |
| | RRT 1.13 | 1.3 | 1.5 | 1.6 | 2.5 | 2.0 | 2.5 |
| | RRT 1.15 | 0.0 | 0.0 | 0.0 | 0.0 | 0.0 | 0.0 |

| | | | | | | |
|---|---|---|---|---|---|---|
| Potency (bioassay) | 111 | 106 | 108 | 113 | 113 | 135 |
| Residual Moisture¹ | 0.2¹ | 0.8 | 0.7 | 0.8 | 1.0 | 0.8 |

For Information Only

| | | | | | | |
|---|---|---|---|---|---|---|
| Purity (IEC-HPLC) | NT | NT | NT | NT | NT | NT |
| Deamidation (pmol/pmol) | NT | NT | NT | NT | NT | 0.0079 |

WC = White Cake,
CPF = Clear, pale yellow, essentially free from foreign particulate matter;
NT = Not tested
¹Result is an average of 3 vials (1 each from beginning, middle, and end)

TABLE 31
Stability Data for Composition 4, 40° C.

| | | Time (months) | | | |
|---|---|---|---|---|---|
| Attributes | | 0 | 1 | 3 | 6 |
| Appearance (Pre-reconstitution) | | WC | WC | WC | WC |
| Appearance (Reconstitution time) | | 1 min | 1 min | 1 min | 1 min |
| Appearance (Post- reconstitution) | | CPF | CPF | CPF | CPF |
| pH | | 7.1 | 7.2 | 7.2 | 7.2 |
| Osmolality (Freezing point) | | 311 | 313 | 320 | 307 |
| Protein Concentration (A₂₈₀) | | 23.6¹ | 23.8 | 24.9 | 23.4 |
| Purity | Reduced | 100 | 99 | 99 | 98 |
| (SDS-PAGE), | Non-reduced | 100 | 99 | 99 | 97 |
| Coomassie | | | | | |
| Purity | Main Peak | 99.1 | 98.1 | 97.7 | 96.5 |
| (SEC-HPLC) | RRT ≦.78 | 0.2 | 0.2 | 0.2 | 0.3 |
| | RRT .86 | 0.4 | 1.0 | 1.7 | 2.7 |
| | RRT 1.09 | 0.3 | 0.7 | 0.4 | 0.5 |
| Purity | Main Peak | 95.3 | 93.6 | 91.9 | 88.9 |
| (RP-HPLC) | RRT .64 | 0.3 | 0.3 | 0.5 | 0.4 |
| | RRT 0.88-0.91 | 0.3 | 0.3 | 0.4 | 0.5 |
| | RRT 0.96-0.99 | 0.2 | 0.4 | 0.6 | 0.9 |
| | RRT 1.03-1.06 | 2.2 | 3.0 | 3.7 | 5.4 |
| | RRT 1.09-1.10 | 0.4 | 0.5 | 0.6 | 0.7 |
| | RRT 1.13 | 1.3 | 1.8 | 2.2 | 3.3 |
| | RRT 1.15 | 0.0 | 0.0 | 0.0 | 0.0 |

| | | | | |
|---|---|---|---|---|
| Potency (bioassay) | 111 | 110 | 121 | 85 |
| Residual Moisture¹ | 0.2¹ | 0.7 | 1.0 | 1.5 |

For Information Only

| | | | | |
|---|---|---|---|---|
| Purity (IEC-HPLC) | NT | NT | NT | NT |
| Deamidation (pmol/pmol) | NT | NT | NT | NT |

WC = White Cake,
CPF = Clear, pale yellow, essentially free from foreign particulate matter;
NT = Not tested
¹Result is an average of 3 vials (1 each from beginning, middle, and end)

Samples stored under recommended conditions are stable under the recommended conditions for 18 months.

Samples stored under recommended conditions are stable for 24 months.

Samples stored under recommended conditions are stable for 36 months.

### Example 6

### Long Term Stability Testing of Composition 5

### Methods

Composition 5, known as Cardeva™, is a recombinant human B-type natriuretic peptide (BNP) serum albumin fusion protein. The TBU lypholization cycle applied to Composition 5 (100 mg/ml) is summarized in Table 32. The lyophilized product was stored at 2-8° C., 25° C. and 40° C.

### Results

At the end of 6 months, there is no significant change in SEC-HPLC purity for Composition 5 when stored at 2-8° C. (Table 33). The quality attributes of samples stored at the recommended conditions meet all acceptance criteria up to 6 months. When stored at elevated temperature conditions, such as 25° C. and 40° C., there is a 0.7% and a 4.0% loss in SEC-HPLC purity after 6 months, respectively (Tables 34-35). However, potency was within tolerances for all temperature conditions up to 6 months (Tables 33, 34 and 35).

TABLE 32
TBU Lyophilization Cycle

| Step | Parameters |
|---|---|
| a | Pre-cool shelves to 5° C. |
| b | Load product and hold for at least 2 hours at 5° C. |
| c | Cool shelf temperature at 0.3° C./min to −45° C. |
| d | Hold for 3 hours at −45° C. |
| e | Warm shelf temperature at 0.8° C./min to −18° C. |
| f | Hold for 5 hours at −18° C. (annealing/thermal treatment). |
| g | Cool shelf temperature −45° C. at 0.3° C./min. |
| h | Hold at −45° C. for 2 hours. |
| i | Engage vacuum and adjust to 100 mT as controlled by capacitance |
| | manometer. |
| j | Hold shelf temperature at −45° C. until vacuum set point is |
| | achieved. |
| k | Hold at −45° C. for 1 hour. |
| l | Warm shelf temperature to −10° C. at 0.8° C./min. |
| m | Hold shelf temperature at −10° C. for 36 hours (primary drying). |
| n | Ensure that all functioning product thermocouples have been at or |
| | above −10° C. for at least 3 hours before proceeding to the next |
| | step. If not, continue to hold the shelf temperature at −10° C. |
| | until all functioning product thermocouples have been at or |
| | above −10° C. for at least 3 hours. |
| o | Warm shelf temperature to 25° C. at 0.8° C./min. |
| p | Hold at 25° C. for 15 hours (secondary drying). |

TABLE 33
Stability Data for Composition 5 When Stored at Recommended Conditions, 2-8° C.

| | | | Time (months) | | | |
|---|---|---|---|---|---|---|
| Attributes | | Acceptance Criteria | 0 | 1 | 3 | 6 |
| Appearance (Visual inspection- | | White to off-white | WC | WC | WC | WC |
| pre-reconstitution; cake) | | cake | | | | |
| Appearance (Visual inspection - | | Report results (min) | ≦4 | ≦3 | ≦6 | ≦3 |
| reconstitution time seconds) | | | | | | |
| Appearance (Visual inspection - | | Clear to opalescent, | CPF | CPF | CPF | CPF |
| post-reconstitution) | | pale yellow to yellow, | | | | |
| | | essentially free from | | | | |
| | | foreign particulate | | | | |
| | | matter | | | | |
| pH | | 6.0 ± 0.4 | 6.0 | 6.0 | 6.0 | |
| Osmolality (Freezing point) | | Report results | 284 | 293 | 325 | 303 |
| | | (mOsm/kg) | | | | |
| Protein Concentration (A₂₈₀) | | 100 ± 15 mg/mL | 102.9 | 100.8 | 112.1 | 99.6 |
| (mg/ml) | | | | | | |
| Purity | Reduced (%) | ≧90.0% | 100 | 100 | 100 | 100 |
| (SDS-PAGE) | Non-reduced (%) | ≧90.0% | 100 | 100 | 100 | 99 |

| | | | | | |
|---|---|---|---|---|---|
| Purity (SEC-HPLC) (%) | ≧90.0% | 98.7 | 98.9 | 98.9 | 99.0 |
| Purity (RP-HPLC) (%) | Report Results (%) | 92.7 | 91.7 | 92.8 | 91.7 |
| Purity (IE-HPLC) (%) | Report Results (%) | 64.9 | 64.6 | 65.1 | 62.8 |
| Potency (bioassay) (relative | Report Results (%) | 30.5 | 34.9 | 28.7 | |
| potency %) | | | | | |
| Residual Moisture (%) | ≦3.0% | 0.03 | 0.03 | 0.06 | 0.9 |
| Free Thiol (Ellman's Reagant) | Report Results | NT | 0.9 | 0.7 | 0.7 |
| | (mol/mol) | | | | |

WC—White cake;
CPF—Clear, pale yellow, essentially free from foreign particulate matter

TABLE 34
Stability Data for Composition 5, 25° C.

| | | Time (months) | | | |
|---|---|---|---|---|---|
| Attributes | | 0 | 1 | 3 | 6 |
| Appearance (Visual inspection - | | WC | WC | WC | WC |
| pre-reconstitution; cake) | | | | | |
| Appearance (Visual inspection - | | ≦4 | ≦4 | ≦7 | ≦3 |
| reconstitution time seconds) | | | | | |
| Appearance (Visual inspection - | | CPF | CPF | CPF | CPF |
| post-reconstitution) | | | | | |
| pH | | 6.0 | 6.1 | 6.0 | 6.1 |
| Osmolality (Freezing point) | | 284 | 303 | 326 | 310 |
| Protein Concentration (A₂₈₀) | | 102.9 | 104.4 | 107.4 | 101.1 |
| (mg/ml) | | | | | |
| Purity | Reduced (%) | 100 | 100 | 100 | 98 |
| (SDS-PAGE) | Non-reduced (%) | 100 | 100 | 100 | 98 |

| | | | | |
|---|---|---|---|---|
| Purity (SEC-HPLC) (%) | 98.7 | 98.4 | 98.1 | 98.0 |
| Purity (RP-HPLC) (%) | 92.7 | 90.9 | 92.5 | 88.8 |
| Purity (IE-HPLC) (%) | 64.9 | 63.3 | 64.1 | 60.9 |
| Potency (bioassay) (relative | 30.5 | 31.8 | 33.9 | |
| potency %) | | | | |
| Free Thiol (Ellman's Reagant) | NT | 0.8 | 0.7 | 0.7 |

WC—White cake;
CPF—Clear, pale yellow, essentially free from foreign particulate matter

TABLE 35
Stability Data for Composition 5, 40° C.

| | | Time (months) | | | |
|---|---|---|---|---|---|
| Attributes | | 0 | 1 | 3 | 6 |
| Appearance (Pre-reconstitution) | | WC | WC | WC | WC |
| Appearance (Reconstitution time) | | ≦4 | ≦4 | ≦6 | ≦3 |
| Appearance (Post- reconstitution) | | CPF | CPF | CPF | CPF |
| pH | | 6.0 | 6.1 | 6.0 | 6.1 |
| Osmolality (Freezing point) | | 284 | 299 | 305 | 308 |
| Protein Concentration (A₂₈₀) | | 102.9 | 103.2 | 114.2 | 99.1 |
| (mg/ml) | | | | | |
| Purity | Reduced (%) | 100 | 100 | 98 | 95 |
| (SDS-PAGE) | Non-reduced (%) | 100 | 100 | 96 | 95 |

| | | | | |
|---|---|---|---|---|
| Purity (SEC-HPLC) (%) | 98.7 | 97.3 | 95.8 | 94.7 |
| Purity (RP-HPLC) (%) | 92.7 | 89.6 | 90.0 | 86.1 |
| Purity (IE-HPLC) (%) | 64.9 | 61.9 | 59.1 | 55.7 |
| Potency (bioassay) (relative | 30.5 | 38.9 | 34.3 | |
| potency %) | | | | |
| Free Thiol (Ellman's Reagant) | NT | 0.9 | 0.7 | 0.8 |

WC—White cake;
CPF—Clear, pale yellow, essentially free from foreign particulate matter

### Discussion

A more concentrated formulation can have significant advantages, including increasing convenience (since fewer or smaller vials are required to contain a given dose) and reducing the injection bolus necessary for a given dose. However, it is not always routine and often very difficult to increase the concentration of a peptide formulation.

The appropriateness of a lyophilization process is unpredictable. Freezing rates that are either too fast or too slow can lead to protein aggregation or denaturing (Rathore and Rajan 2008; Krishnamurthy and Manning 2002). Excessive drying can destabilize the protein (Rathore and Rajan 2008). Even the material used for the vial and the stopper can have critical effect on lyophilized protein products (Rathore and Rajan 2008).

As the concentration of protein in the solution used to make a lyophilized product increases, the time required to reconstitute the lyophilate increases as well (Shire et al. 2004).

The process described herein, however, represents an approach which produces pharmaceutically acceptable lyophilized cakes with fast reconstitution times.

### REFERENCES

Leader, Benjamin, Quentin J. Baca, and David E. Golan. “Protein therapeutics: a summary and pharmacological classification.” Nature Reviews Drug Discovery 7.1 (2008): 21-39.
Rathore, Nitin, and Rahul S. Rajan. “Current perspectives on stability of protein drug products during formulation, fill and finish operations.” Biotechnology Progress 24.3 (2008): 504-514.
Shire, Steven J., Zahra Shahrokh, and Jun Liu. “Challenges in the development of high protein concentration formulations.” Journal of Pharmaceutical Sciences 93.6 (2004): 1390-1402.

1. A process for producing a lyophilized pharmaceutical composition containing a protein, comprising the steps of:
(i) obtaining a solution comprising the protein in one or more containers;
(ii) placing the one or more containers within a chamber of a lyophilizing unit;
(iii) reducing the temperature to an initial freezing temperature of −60° C. to −25° C. at a rate of 0.2° C. to 2.0° C. per minute, and holding the temperature at the initial freezing temperature for 1 to 6 hours to form a frozen solution;
(iv) increasing the temperature to an annealing temperature of the frozen solution of −30° C. to −10° C. at a rate of 0.2° C. to 2.0° C. per minute, and holding the temperature at the annealing temperature for 1 to 10 hours;
(v) reducing the temperature to a refreezing temperature of −60° C. to −25° C. at a rate of 0.2° C. to 2.0° C. per minute, and holding the temperature at the refreezing temperature for 1 to 6 hours;
(vi) reducing the pressure of the chamber to 50 to 500 mT, and continuing to hold the temperature at the refreezing temperature for an additional 0 to 4 hours;
(vii) increasing the temperature to a primary drying temperature of −30° C. to −5° C. at a rate of 0.2° C. to 2.0° C. per minute, and holding the temperature at the primary drying temperature for 10 to 72 hours;
(viii) increasing the temperature to a secondary drying temperature of 5° C. to 30° C. at a rate of 0.2° C. to 2.0° C. per minute, and holding the temperature at the secondary drying temperature for 2 to 25 hours; and
(ix) increasing the pressure of the chamber to partial atmospheric pressure.

2. The process of claim 1, wherein step (ii) placing the containers within the chamber of the lyophilizing unit comprises placing the containers on a shelf which is at an initial shelf temperature of from −40° C. to 10° C. within the chamber and holding the temperature of the shelf at the initial shelf temperature for 0 to 5 hours before initiating step (iii).

3-7. (canceled)

8. The process of claim 2, wherein the shelf is held at the initial shelf temperature for 2 hours or more.

9. (canceled)

10. The process of claim 1, wherein step (ii) further comprises pre-cooling the one or more containers.

11-13. (canceled)

14. The process of claim 1, wherein in step (iii) the temperature is reduced at a rate of 0.3° C. per minute.

15. (canceled)

16. (canceled)

17. The process of claim 1, wherein in step (iii) the temperature is held at the initial freezing temperature for 3 hours.

18. The process of claim 1, wherein in step (iv) the temperature is increased at a rate of 0.8° C. per minute.

19. (canceled)

20. The process of claim 1, wherein in step (iv) the temperature is held at the annealing temperature for 5 hours.

21. The process of claim 1, wherein in step (v) the temperature is reduced at a rate of 0.3° C. per minute.

22-24. (canceled)

25. The process of claim 1, wherein in step (vii) the temperature is increased at a rate of 0.6° C. per minute.

26-30. (canceled)

31. The process of claim 1, further comprising measuring the temperature of the frozen solution within one or more of the containers during step (vii), wherein in step (vii) the temperature is held at the primary drying temperature for three hours beyond the time at which the temperature of each measured container is equal to or greater than the primary drying temperature.

32. The process of claim 1, wherein in step (viii) the temperature is increased at a rate of 0.6° C. per minute.

33-38. (canceled)

39. The process of claim 1, further comprising the step:
(x) sealing the containers.

40-62. (canceled)

63. The process of claim 1, wherein the solution comprising the protein further comprises 100 to 150 mM mannitol, 20 to 40 mM trehalose, or 0.02 to 0.05 percent polysorbate 80.

64-72. (canceled)

73. The process of claim 1 for producing a lyophilized pharmaceutical composition containing a protein, wherein the protein is Composition 1.

74. (canceled)

75. (canceled)

76. The process of claim 1 for producing a lyophilized pharmaceutical composition containing a protein, wherein the protein is a human serum albumin fusion protein.

77. A product produced by the process of claim 1.

78-82. (canceled)

83. A process for producing an injectable pharmaceutical composition, comprising obtaining an amount of the lyophilized pharmaceutical composition comprising a protein produced by the process of claim 1, and reconstituting the lyophilized pharmaceutical composition with water for injection within 15 minutes, thereby producing an injectable pharmaceutical composition.

84. A method of treating a patient with a therapeutic protein composition, comprising obtaining an amount of the lyophilized pharmaceutical composition comprising a protein produced by the process of claim 1, reconstituting the lyophilized pharmaceutical composition with water for injection within 15 minutes to form a reconstituted solution, and administering the reconstituted solution to the patient, thereby treating the patient.

85. (canceled)

86. (canceled)

87. A process for producing a lyophilized pharmaceutical composition containing a protein, comprising the steps of:
(i) obtaining a solution comprising the protein in one or more containers;
(ii) placing the one or more containers within a chamber of a lyophilizing unit;
(iii) reducing the temperature to an initial freezing temperature of −60° C. to −25° C. at a rate of 0.2° C. to 2.0° C. per minute, and holding the temperature at the initial freezing temperature for 1 to 6 hours to form a frozen solution;
(iv) reducing the pressure of the chamber to 50 to 500 mT, and continuing to hold the temperature at the freezing temperature for an additional 0 to 4 hours;
(v) increasing the temperature to a primary drying temperature of −30° C. to −5° C. at a rate of 0.2° C. to 2.0° C. per minute, and holding the temperature at the primary drying temperature for 10 to 72 hours; and
(vi) increasing the temperature to a secondary drying temperature of 5° C. to 30° C. at a rate of 0.2° C. to 2.0° C. per minute, and holding the temperature at the secondary drying temperature for 2 to 25 hours.

88-90. (canceled)
